(19) United States
(12) Patent Application Publication
(10) Pub. No.: US 2023/0168252 A1
(43) Pub. Date: Jun. 1, 2023

(54) DECTECTION OF EXOSOMES AND EXOSOMAL BIOMARKERS FOR THE DIAGNOSIS AND PROGNOSIS OF DISEASES AND DISORDERS

(71) Applicant: NANOSOMIX, INC., Aliso Viejo, CA (US)

(72) Inventor: Masato Mitsuhashi, Irvine, CA (US)

(21) Appl. No.: 17/958,264

(22) Filed: Sep. 30, 2022

Related U.S. Application Data
(63) Continuation of application No. 16/252,537, filed on Jan. 18, 2019, now abandoned.
(60) Provisional application No. 62/619,080, filed on Jan. 18, 2018.

(51) Int. Cl. G01N 33/574; G01N 33/543; G01N 33/68; G01N 33/553; G01N 33/552

(52) U.S. Cl. CPC G01N 33/574; G01N 33/54326; G01N 33/6896; G01N 33/553; G01N 33/5432; G01N 33/54346; G01N 33/552; G01N 33/54306; G01N 33/543

(57) ABSTRACT

The present invention relates to methods, compositions, and kits for detecting and quantitating exosomes and exosomal biomarkers and the use of exosomes and exosomal biomarkers in diagnostic and prognostic methods for various diseases and disorders. Disease and disorders of the present invention include neurological disorders, immunological disorders, placental diseases, cancer, hematological disorders, kidney disease, gastrointestinal diseases, liver diseases, and musculoskeletal diseases

### RELATED APPLICATIONS

This application is a continuation of U.S. patent application Ser. No. 16/252,537, filed on Jan. 18, 2019, which claims priority to the U.S. Provisional Patent Application Ser. No. 62/619,080, filed on Jan. 18, 2018, which is hereby incorporated by reference herein in their entirety.

### FIELD OF THE INVENTION

The present invention relates to methods, compositions, and kits for detecting and quantitating exosomes and exosomal biomarkers and the use of exosomes and exosomal biomarkers in diagnostic and prognostic methods for various diseases and disorders. Disease and disorders of the present invention include neurological disorders, immunological disorders, placental diseases, cancer, hematological disorders, kidney disease, gastrointestinal diseases, liver diseases, and musculoskeletal diseases.

### BACKGROUND OF THE INVENTION

Exosomes in biological fluids are potentially useful diagnostically for various diseases, because exosomes carry physiological and pathological materials (proteins, metabolites, RNAs, small molecules, etc.) of the mother cells from which they originate and the microenvironment near their mother cells. However, even though exosomes are recognized as valuable resources for diagnostics, current analytical methods of analyzing exosomes are too complicated and expensive for routine use in diagnostic testing.

More than 5.4 million Americans and 35 million people worldwide have Alzheimer's disease, the most common form of dementia. Currently, the only definitive way to diagnose Alzheimer's disease is by direct examination of brain tissue after a patient dies. Doctors use brain imaging, evaluation of behavior, psychiatric tests, and other means to diagnose the disease in the patients suspected of having Alzheimer's disease, but none are highly accurate, and many are costly or not practical.

In 2017, there will be an estimated 1,688,780 new cancer cases diagnosed and 600,920 cancer deaths in the US. The most common cancers are breast cancer, lung and bronchus cancer, prostate cancer, colon and rectum cancer, bladder cancer, melanoma of the skin, non-Hodgkin lymphoma, thyroid cancer, kidney and renal pelvis cancer, leukemia, endometrial cancer, and pancreatic cancer. Similarly, millions of Americans suffer from immunological disorders and diseases caused by a dysfunction of the immune system.

Therefore, there is a need in the art for biomarkers and methods for diagnosing diseases and other disorders, such as, for example, neurological disorders, cancer, immunological disorders, and placental disease. Additionally, there is a need in the art for compositions for detecting exosomes and exosomal biomarkers as well as compositions and methods useful for treating diseases and other disorders. The present invention meets this need by providing novel methods for detecting exosomes and exosomal biomarkers. The present invention further provides methods, assays, biomarkers, kits, and compositions for diagnosing, prognosing, predicting, and treating various diseases and disorders.

### SUMMARY OF THE INVENTION

The present invention relates to novel methods, compositions, and kits for detecting and quantitating vesicles (e.g., exosomes) and vesicle biomarkers. In some embodiments, the invention provides a method comprising: a) providing a biological sample comprising vesicles; b) contacting a solid support comprising capture agents associated therewith with the biological sample under conditions wherein the capture agents selectively bind to the vesicles, thereby capturing said vesicles on the solid support; c) lysing or permeabilizing the vesicles while maintaining contact between the vesicle and the solid support, and between vesicle membrane-bound biomarker and vesicle membrane; and d) isolating vesicle core from the captured vesicles. In some embodiments, the biological sample is selected from the group consisting of whole blood, serum, plasma, urine, interstitial fluid, peritoneal fluid, tears, saliva, cerebrospinal fluid, cell and/or bacterial culture supernatants, cervical swab, buccal swab, tissues, organs, and environmental materials. In other embodiments, the vesicles are selected from the group consisting of exosomes, microparticles, microvesicles, nanosomes, extracellular vesicles, ectosomes, and apoptotic bodies. In yet other embodiments, the exosomes are selected from the group consisting of neuron-derived exosomes, astrocyte-derived exosomes, oligodendrocyte-derived exosomes, microglia-derived exosomes, dopaminergic neuron-derived exosomes, cholinergic neuron-derived exosomes, serotonergic neuron-derived exosomes, histaminergic neuron-derived exosomes, glutaminergic neuron-derived exosomes, glycinergic neuron-derived exosomes, adrenergic neuron-derived exosomes, gabaergic neuron-derived exosomes, opipoidergic neuron-derived exosomes, cancer-derived exosomes, bone marrow-derived exosomes, lymph node-derived exosomes, prostate-derived exosomes, lung-derived exosomes, liver-derived exosomes, pancreas-derived exosomes, ovary-derived exosomes, gastrointestinal-derived exosomes, kidney and urinary tract-derived exosomes, skin-derived exosomes, bone-derived exosomes, muscle-derived exosomes, peripheral nerve-derived exosomes, adipose tissue-derived exosomes, connective tissue-derived exosomes, male or female organ-derived exosomes, spleen-derived exosomes, endocrine-derived exosomes, and vascular-derived exosomes. In still other embodiments, the solid support is a plate, a non-magnetic bead, a magnetic bead, a filter, a slide, a wafer, a rod, a particle, a strand, a disc, a membrane, or a surface of a tube, channel, column, flow cell device, pipette tip, or microfluidic device. In some embodiments, the solid support comprises glass, quartz, silicon, metal, ceramic, plastic, nylon, polyacrylamide, agarose, a hydrogel, or a resin.

In some embodiments, the capture agents used in the methods of the present invention are antibodies, antibody fragments, antibody mimetics, aptamers, receptors, or ligands that specifically bind to the vesicles. In other embodiments, the antibodies used in the methods of the present invention are monoclonal or polyclonal antibodies against CD81, CD63, CD9, CD171, NCAM, SNAP25, EAAT1, OMG, CD11b, Tau, amyloid beta, alpha-synuclein, TDP-43, and/or SOD. In still other embodiments, the methods of the invention further comprise detecting one or more cargo and/or cytosolic biomarkers from the vesicle core. In other embodiments, the biomarker is a mutated protein, an over- or under expressed protein, a modified protein, an organ/tissue-specific protein, a disease-associated protein, a protein monomer and/or oligomer, an enzyme, a kinase, a hormone, a growth factor, a transcription factor, a cytokine and/or chemokine, miRNA, mRNA, non-coding RNA, a neurotransmitter, a small molecule, a metabolite, a lipid and/or lipoprotein, a metal, a foreign body, and/or control markers. In other embodiments, the modified protein is phosphorylated protein, methylated protein, glycosylated protein, acetylated protein, or ubiquitinated protein. In yet other embodiments, the disease-associated proteins are selected from the group consisting of a monomer, oligomer, or phosphorylated form of tau, amyloid beta, alpha-synuclein, TDP-43, SOD. In other embodiments, the control markers comprise total protein, synaptic proteins, GAPDH, GFAP, and MBP. In some embodiments, the detection comprise immunoassay, Western blot, Northern blot, chromatography, mass-spectrometry, sequencing, and/or reaction with nucleic acid dye. In yet other embodiments, the biological sample is obtained from a subject who has been diagnosed or is suspected of having a neurological disorder, Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, Parkinson's disease. In still other embodiments, the biological sample is obtained from a subject who has been diagnosed or is suspected of having breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors. In some embodiments, the methods of the present invention further comprise treating the subject for a disease or disorder if the subject is diagnosed as having the disease or disorder.

In some embodiments, the present invention provides a method comprising: a) providing a biological sample comprising exosomes; b) contacting a solid support comprising capture agents associated therewith with the biological sample under conditions wherein the capture agents selectively bind to the exosomes, thereby capturing said exosomes on the solid support; c) lysing or permeabilizing the exosomes while maintaining contact between the exosome membrane and the capture agents; and d) detecting inner membrane-bound biomarkers from the exosomes. In some embodiments, the inner membrane-bound biomarker is selected from the group consisting of a monomer, oligomer, phosphorylated form of tau, synaptophysin, synaptotagmin, synaptopodin, SNAP25, neurofilament, amyloid beta, alpha-synuclein, TDP-43, SOD aptic protein, a cytoskeletal protein, a membrane receptor associated protein and/or kinase. In other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes, cancer-derived exosomes, bone marrow-derived exosomes, lymph node-derived exosomes, prostate-derived exosomes, lung-derived exosomes, liver-derived exosomes, pancreas-derived exosomes, ovary-derived exosomes, gastrointestinal-derived exosomes, kidney and urinary tract-derived exosomes, skin-derived exosomes, bone-derived exosomes, muscle-derived exosomes, peripheral nerve-derived exosomes, adipose tissue-derived exosomes, connective tissue-derived exosomes, male or female organ-derived exosomes, spleen-derived exosomes, endocrine-derived exosomes, and vascular-derived exosomes.

In some embodiments, the invention provides a method comprising the steps of: a) providing a biological sample comprising exosomes; b) contacting a solid support comprising capture agents associated therewith with the biological sample under conditions wherein the capture agents selectively bind to the exosomes, thereby capturing said exosomes on the solid support; c) lysing or permeabilizing the exosomes while maintaining contact between the exosome and the solid support, and between exosome membrane-bound biomarker and exosome membrane; and d) isolating exosome core and/or cargo from the solid support. In some embodiments, the methods further comprises detecting cargo and/or cytosolic biomarkers from the exosome core. In some embodiments, the biomarker is a mutated protein, an over- or under expressed protein, a modified protein (e.g., phosphorylated protein, methylated protein, glycosylated protein, acetylated protein, or ubiquitinated protein), an organ/tissue-specific protein (e.g., neurofilaments for neuron, GFAP for astrocytes, surfactant proteins for lung, PSA for prostate), a disease-specific protein (e.g., Tau or Abeta), a protein monomer and/or oligomer, an enzyme, a kinase, a hormone, a growth factor, a transcription factor, a cytokine and/or chemokine, an RNA (e.g., miRNA, mRNA, and/or non-coding RNA), a neurotransmitter (e.g., acetylcholine, dopamine, serotonin, opioid), a small molecule (e.g. drug or nitric oxide), a metabolite, a lipid and/or lipoprotein, a metal, or a foreign body (e.g., bacteria and/or viral origin biomolecules). In some embodiments, the biomarker is Tau monomers and/or oligomers. In other embodiments the biomarker is alpha-synuclein. In yet other embodiments, the biomarker is phosphorylated-Tau or Abeta monomers and/or oligomers. In still other embodiments, the biomarker is TDP-43 or SOD. In some embodiments, the solid support is a plate, a non-magnetic bead, a magnetic bead, a filter, a slide, a wafer, a rod, a particle, a strand, a disc, a membrane, or a surface of a tube, channel, column, flow cell device, or microfluidic device. In other embodiments, the solid support comprises glass, quartz, silicon, metal, ceramic, plastic, nylon, polyacrylamide, a hydrogel, or a resin. In yet other embodiments, the solid support comprises at least 2, 3, 4, 5, 6, 7, 8, 9, 10, 15, 20, or more different capture agents that selectively bind to the exosomes or inner membrane-bound exosomal biomarkers. In still other embodiments, the capture agents comprise antibodies, antibody fragments, antibody mimetics, or aptamers that specifically bind to the exosomes. In some embodiments, detection of the one or more cargo or cytosolic biomarkers from the exosome core comprises using one or more detection agents. In other embodiments, the detection agents comprise antibodies, antibody fragments, antibody mimetics, or aptamers that specifically bind to the inner membrane-bound biomarkers from the exosomes. In yet other embodiments, the detection agent further comprises a detectable label. In still other embodiments, the detectable label is a fluorescent label, a chemiluminescent label, an electrochemiluminescent label, a bioluminescent label, an isotopic label, or a radioactive label. In some embodiments, the detecting a cargo or cytosolic biomarker from an exosome core comprises performing an immunoassay. In other embodiments, the immunoassay is an enzyme-linked immunosorbent assay (ELISA), an immunofluorescent assay (IFA), an immune-polymerase chain reaction assay, an electro-chemiluminescence immunoassay (ECLIA), or a radioimmunoassay (RIA). In still other embodiments, the biological sample comprising exosomes is from a subject or a cell culture supernatant. In yet other embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In some embodiments, the biological sample is obtained from a subject who has been diagnosed or is suspected of having a neurological disorder, such as Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, or Parkinson's disease. In some embodiments, a biological sample is obtained from a subject who has been diagnosed or is suspected of having cancer, such as breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors. In other embodiments, the biological sample is obtained from a subject who has been diagnosed or is suspected of having an immunological disorder. In yet other embodiments, the biological sample is obtained from a subject who has been diagnosed or is suspected of having a placental disease. In still other embodiments, the biological sample is obtained from a subject who has been diagnosed or is suspected of having a hematological disorder, a kidney disease, a gastrointestinal disease, a liver disease, or a musculoskeletal disease. In some embodiments, the subject is selected from the group consisting of a human, a monkey, a dog, a pig, a bovine, a rabbit, a guinea pig, and a rodent. In other embodiments, the methods of the present invention further comprise diagnosing the subject with a disease or disorder. In other embodiments, the methods of the present invention further comprise treating the subject for a disease or disorder if the subject is diagnosed as having the disease or disorder.

In some embodiments, the invention provides a method for selectively capturing exosomes on a solid support, lysing or permeabilizing the exosomes while maintaining contact between the exosome membrane and the solid support, and detecting inner membrane-bound biomarkers from the captured exosomes. In other embodiments, the invention provides a method for selectively capturing exosomes on a solid support, lysing or permeabilizing the exosomes while maintaining contact between the exosome membrane and the solid support, and detecting cargo or cytosolic biomarkers from the captured exosomes. In yet other embodiments, the invention provides a method for selectively capturing exosomes on a solid support, lysing or permeabilizing the exosomes while maintaining contact between the exosome membrane and the solid support, and detecting miRNA from the captured exosomes. In still other embodiments, the invention provides saturation enzyme-linked immunosorbent assays for detecting exosomes and exosomal biomarkers. In certain embodiments, the assay comprises incubating a sample comprising exosomes on a solid support comprising immobilized antibodies, wherein the immobilized antibodies on the solid support are generally saturated by the exosomes in the sample. In certain embodiments, substantially all of the immobilized antibodies are bound to a target antigen from the sample. In certain aspects of the present embodiment, the method further comprises, lysing or permeabilizing the exosomes while maintaining contact between the exosome membrane and the solid support, and detecting cargo biomarkers or inner-membrane bound biomarkers from the captured exosomes.

Measurement of exosomes and exosomal biomarkers is useful in diagnostic and prognostic methods for various diseases. In particular, the present invention provides methods of diagnosing or prognosing a disease or disorder in a subject, identifying a subject at risk of a disease or a disorder, or prescribing a therapeutic regimen or predicting benefit from therapy in a subject having a disease or a disorder. In certain embodiments, the present invention provides methods of diagnosing or prognosing a neurological disorder in a subject, identifying a subject at risk of a neurological disorder, or prescribing a therapeutic regimen or predicting benefit from therapy in a subject having a neurological disorder. In other embodiments, the present invention provides methods of diagnosing or prognosing an immunological disorder in a subject, identifying a subject at risk of an immunological disorder, or prescribing a therapeutic regimen or predicting benefit from therapy in a subject having an immunological disorder. In other embodiments, the present invention provides methods of diagnosing or prognosing cancer in a subject, identifying a subject at risk of cancer, or prescribing a therapeutic regimen or predicting benefit from therapy in a subject having cancer. In other embodiments, the present invention provides methods of diagnosing or prognosing placental disease in a subject, identifying a subject at risk of a placental disease, or prescribing a therapeutic regimen or predicting benefit from therapy in a subject having a placental disease. In other embodiments, the present invention provides methods of diagnosing or prognosing hematological disorders in a subject, identifying a subject at risk of hematological disorders, or prescribing a therapeutic regimen or predicting benefit from therapy in a subject having hematological disorders. In other embodiments, the present invention provides methods of diagnosing or prognosing kidney disease in a subject, identifying a subject at risk of kidney disease, or prescribing a therapeutic regimen or predicting benefit from therapy in a subject having kidney disease. In other embodiments, the present invention provides methods of diagnosing or prognosing gastrointestinal disease in a subject, identifying a subject at risk of gastrointestinal disease, or prescribing a therapeutic regimen or predicting benefit from therapy in a subject having gastrointestinal disease. In other embodiments, the present invention provides methods of diagnosing or prognosing liver disease in a subject, identifying a subject at risk of liver disease, or prescribing a therapeutic regimen or predicting benefit from therapy in a subject having liver disease. In other embodiments, the present invention provides methods of diagnosing or prognosing musculoskeletal disease in a subject, identifying a subject at risk of musculoskeletal disease, or prescribing a therapeutic regimen or predicting benefit from therapy in a subject having musculoskeletal disease.

In one aspect, the invention provides a method comprising: a) providing a biological sample comprising exosomes; b) contacting a solid support comprising capture agents associated therewith with the biological sample under conditions wherein the capture agents selectively bind to the exosomes, thereby capturing said exosomes on the solid support; c) lysing or permeabilizing the exosomes while maintaining contact between the exosome membrane and the capture agents; and d) detecting inner membrane-bound biomarkers from the exosomes. In some embodiments, the inner membrane-bound biomarker is selected from the group consisting of a monomer and/or oligomer tau, phosphorylated tau, a synaptic protein, a cytoskeletal protein, and a membrane receptor associated protein and/or kinase. In some embodiments, the synaptic protein is synaptophysin, synaptotagmin, synaptopodin, or SNAP25. In other embodiments, the cytoskeletal protein is a neurofilament. In other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. In certain embodiments, the solid support is a plate, a non-magnetic bead, a magnetic bead, a filter, a slide, a wafer, a rod, a particle, a strand, a disc, a membrane, or a surface of a tube, channel, column, flow cell device, or microfluidic device. The solid support can comprise, for example, glass, quartz, silicon, metal, ceramic, plastic, nylon, polyacrylamide, a hydrogel, or a resin. In certain embodiments, the solid support comprises more than one type of capture agent associated therewith, for example, at least 2, 3, 4, 5, 6, 7, 8, 9, 10, 15, 20, or more different capture agents that selectively bind to the exosomes or inner membrane-bound exosomal biomarkers. In certain embodiments, detection of the inner membrane-bound biomarkers from the exosomes captured on the solid support comprises using more than one type of detection agent, for example, at least 2, 3, 4, 5, 6, 7, 8, 9, 10, 15, 20, or more different detection agents that selectively bind to different inner membrane-bound biomarkers from the exosomes. In certain embodiments, the capture agents comprise antibodies, antibody fragments, antibody mimetics, or aptamers that specifically bind to the exosomes. In other embodiments, the detection agents comprise antibodies, antibody fragments, antibody mimetics, or aptamers that specifically bind to the inner membrane-bound biomarkers from the exosomes. Capture agents and detection agents may comprise monoclonal antibodies, polyclonal antibodies, chimeric antibodies, nanobodies, recombinant fragments of antibodies, Fab fragments, Fab′ fragments, F(ab′)₂ fragments, Fᵥ fragments, or scFᵥ fragments. In certain embodiments, the detection agent further comprises a detectable label, for example, a fluorescent, chemiluminescent, electrochemiluminescent, bioluminescent, isotopic, or radioactive label. In certain embodiments, detecting an inner membrane-bound biomarker from an exosome comprises performing an immunoassay, such as an enzyme-linked immunosorbent assay (ELISA), an immunofluorescent assay (IFA), an immune-polymerase chain reaction assay, an electro-chemiluminescence immunoassay (ECLIA), or a radioimmunoassay (RIA). The biological sample comprising exosomes may be from a subject or a cell culture supernatant. In certain embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In certain embodiments, a biological sample is obtained from a subject who has been diagnosed or is suspected of having a neurological disorder, such as Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, or Parkinson's disease. In some embodiments, a biological sample is obtained from a subject who has been diagnosed or is suspected of having cancer, such as breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors. In some embodiments, a biological sample is obtained from a subject who has been diagnosed or is suspected of having an immunological disorder. In other embodiments, a biological sample is obtained from a subject who has been diagnosed or is suspected of having a placental disease. In some embodiments, a biological sample is obtained from a subject who has been diagnosed or is suspected of having a hematological disorder, a kidney disease, a gastrointestinal disease, a liver disease, or a musculoskeletal disease. In other embodiments, the subject is selected from the group consisting of a human, a monkey, a dog, a pig, a bovine, a rabbit, a guinea pig, and a rodent. In some embodiments, the methods further comprise diagnosing the subject with a disease or disorder. In other embodiments, the methods further comprise treating the subject for a disease or disorder if the subject is diagnosed as having the disease or disorder.

In one aspect, the invention provides a method comprising: a) providing a biological sample comprising exosomes; b) contacting a solid support comprising capture agents associated therewith with the biological sample under conditions wherein the capture agents selectively bind to the exosomes, thereby capturing said exosomes on the solid support; c) lysing or permeabilizing the exosomes while maintaining contact between the exosome membrane and the capture agents; and d) detecting cargo or cytosolic biomarkers from the exosomes. In some embodiments, the cargo or cytosolic biomarker is selected from the group consisting of neurotransmitters and their metabolites, various enzymes including neurotransmitter synthesis and degradation, synaptic proteins, cytoskeletons, cytokines, and chemokines. In some embodiments, the cargo or cytosolic biomarker is α-synuclein, β-amyloid, tau or phosphorylated tau. In other embodiments, the neurotransmitters are selected from the group consisting of acetylcholine, epinephrine, norepinephrine, dopamine, serotonin, histamine, glutamine, gamma-Aminobutyric acid (GABA), N-Methyl-D-aspartate (NMDA), and opioids. In other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. In certain embodiments, the solid support is a plate, a non-magnetic bead, a magnetic bead, a filter, a slide, a wafer, a rod, a particle, a strand, a disc, a membrane, or a surface of a tube, channel, column, flow cell device, or microfluidic device. The solid support can comprise, for example, glass, quartz, silicon, metal, ceramic, plastic, nylon, polyacrylamide, a hydrogel, or a resin. In certain embodiments, the solid support comprises more than one type of capture agent associated therewith, for example, at least 2, 3, 4, 5, 6, 7, 8, 9, 10, 15, 20, or more different capture agents that selectively bind to the exosomes or cargo or cytosolic exosomal biomarkers. In certain embodiments, detection of the cargo or cytosolic biomarkers from the exosomes captured on the solid support comprises using more than one type of detection agent, for example, at least 2, 3, 4, 5, 6, 7, 8, 9, 10, 15, 20, or more different detection agents that selectively bind to different cargo or cytosolic biomarkers from the exosomes. In certain embodiments, the capture agents comprise antibodies, antibody fragments, antibody mimetics, or aptamers that specifically bind to the exosomes. In other embodiments, the detection agents comprise antibodies, antibody fragments, antibody mimetics, or aptamers that specifically bind to the cargo or cytosolic biomarkers from the exosomes. Capture agents and detection agents may comprise monoclonal antibodies, polyclonal antibodies, chimeric antibodies, nanobodies, recombinant fragments of antibodies, Fab fragments, Fab′ fragments, F(ab′)₂ fragments, Fᵥ fragments, or scFᵥ fragments. In certain embodiments, the detection agent further comprises a detectable label, for example, a fluorescent, chemiluminescent, electrochemiluminescent, bioluminescent, isotopic, or radioactive label. In certain embodiments, detecting a cargo or cytosolic biomarker from an exosome comprises performing an immunoassay, such as an enzyme-linked immunosorbent assay (ELISA), an immunofluorescent assay (IFA), an immune-polymerase chain reaction assay, an electro-chemiluminescence immunoassay (ECLIA), or a radioimmunoassay (RIA). The biological sample comprising exosomes may be from a subject or a cell culture supernatant. In certain embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In certain embodiments, a biological sample is obtained from a subject who has been diagnosed or is suspected of having a neurological disorder, such as Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, or Parkinson's disease. In some embodiments, a biological sample is obtained from a subject who has been diagnosed or is suspected of having cancer, such as breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors. In some embodiments, a biological sample is obtained from a subject who has been diagnosed or is suspected of having an immunological disorder. In other embodiments, a biological sample is obtained from a subject who has been diagnosed or is suspected of having a placental disease. In some embodiments, a biological sample is obtained from a subject who has been diagnosed or is suspected of having a hematological disorder, a kidney disease, a gastrointestinal disease, a liver disease, or a musculoskeletal disease. In other embodiments, the subject is selected from the group consisting of a human, a monkey, a dog, a pig, a bovine, a rabbit, a guinea pig, and a rodent. In some embodiments, the methods further comprise diagnosing the subject with a disease or disorder. In other embodiments, the methods further comprise treating the subject for a disease or disorder if the subject is diagnosed as having the disease or disorder.

In one aspect, the invention provides a method comprising: a) providing a biological sample comprising exosomes; b) contacting a solid support comprising capture agents associated therewith with the biological sample under conditions wherein the capture agents selectively bind to the exosomes, thereby capturing said exosomes on the solid support; c) lysing or permeabilizing the exosomes while maintaining contact between the exosome membrane and the capture agents; and d) detecting miRNA from the exosomes. In other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. In certain embodiments, the solid support is a plate, a non-magnetic bead, a magnetic bead, a filter, a slide, a wafer, a rod, a particle, a strand, a disc, a membrane, or a surface of a tube, channel, column, flow cell device, or microfluidic device. The solid support can comprise, for example, glass, quartz, silicon, metal, ceramic, plastic, nylon, polyacrylamide, a hydrogel, or a resin. In certain embodiments, the solid support comprises more than one type of capture agent associated therewith, for example, at least 2, 3, 4, 5, 6, 7, 8, 9, 10, 15, 20, or more different capture agents that selectively bind to the exosomes or exosomal miRNA. In some embodiments, the methods further comprise incubation of exosomes with Trizol. In certain embodiments, detection of the miRNA from the exosomes captured on the solid support comprises using more than one type of detection agent, for example, at least 2, 3, 4, 5, 6, 7, 8, 9, 10, 15, 20, or more different detection agents that selectively bind to different miRNA from the exosomes. In certain embodiments, the capture agents comprise antibodies, antibody fragments, antibody mimetics, or aptamers that specifically bind to the exosomes. In other embodiments, the detection agents comprise antibodies, antibody fragments, antibody mimetics, or aptamers that specifically bind to the miRNA from the exosomes. In some embodiments, the method further comprises isolating miRNA from the sample by ethanol extraction, magnetic bead separation, or spin column extraction. In some embodiments, the method further comprises determining the level of the miRNA comprising amplifying the miRNA, sequencing the RNA, hybridization, or a gene chip. Capture agents and detection agents may comprise monoclonal antibodies, polyclonal antibodies, chimeric antibodies, nanobodies, recombinant fragments of antibodies, Fab fragments, Fab′ fragments, F(ab′)₂ fragments, Fᵥ fragments, or scFᵥ fragments. In certain embodiments, the detection agent further comprises a detectable label, for example, a fluorescent, chemiluminescent, electrochemiluminescent, bioluminescent, isotopic, or radioactive label. In certain embodiments, detecting miRNA from an exosome comprises performing an immunoassay, such as an enzyme-linked immunosorbent assay (ELISA), an immunofluorescent assay (IFA), an immune-polymerase chain reaction assay, an electro-chemiluminescence immunoassay (ECLIA), or a radioimmunoassay (RIA). The biological sample comprising exosomes may be from a subject or a cell culture supernatant. In certain embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In certain embodiments, a biological sample is obtained from a subject who has been diagnosed or is suspected of having a neurological disorder, such as Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, or Parkinson's disease. In some embodiments, a biological sample is obtained from a subject who has been diagnosed or is suspected of having cancer, such as breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors. In some embodiments, a biological sample is obtained from a subject who has been diagnosed or is suspected of having an immunological disorder. In other embodiments, a biological sample is obtained from a subject who has been diagnosed or is suspected of having a placental disease. In some embodiments, a biological sample is obtained from a subject who has been diagnosed or is suspected of having a hematological disorder, a kidney disease, a gastrointestinal disease, a liver disease, or a musculoskeletal disease. In other embodiments, the subject is selected from the group consisting of a human, a monkey, a dog, a pig, a bovine, a rabbit, a guinea pig, and a rodent. In some embodiments, the methods further comprise diagnosing the subject with a disease or disorder. In other embodiments, the methods further comprise treating the subject for a disease or disorder if the subject is diagnosed as having the disease or disorder.

In other embodiments, the present invention provides a saturation enzyme-linked immunosorbent assay, the assay comprising incubating a sample comprising vesicles on a solid support comprising immobilized antibodies, wherein the immobilized antibodies on the solid support are generally saturated by the vesicles in the sample. In some embodiments, substantially all of the immobilized antibodies are bound to a target antigen from the sample. In other embodiments, the methods further comprise lysing or permeabilizing the vesicles while maintaining contact between the vesicle membrane and the solid support, and detecting cargo or cytosolic biomarkers or inner-membrane bound biomarkers from the captured vesicles. In yet other embodiments, the cargo or cytosolic biomarker or inner-membrane bound biomarker is selected from the group consisting of neurotransmitters and their metabolites, various enzymes including neurotransmitter synthesis and degradation, synaptic proteins, cytoskeletons, cytokines, chemokines, a monomer and/or oligomer tau, phosphorylated tau, a synaptic protein, a cytoskeletal protein, and a membrane receptor associated protein and/or kinase. In still other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. In some embodiments, the vesicles are selected from the group consisting of exosomes, microparticles, microvesicles, nanosomes, extracellular vesicles, ectosomes, and apoptotic bodies. In other embodiments, the exosomes are selected from the group consisting of neuron-derived exosomes, astrocyte-derived exosomes, oligodendrocyte-derived exosomes, microglia-derived exosomes, dopaminergic neuron-derived exosomes, cholinergic neuron-derived exosomes, serotonergic neuron-derived exosomes, histaminergic neuron-derived exosomes, glutaminergic neuron-derived exosomes, glycinergic neuron-derived exosomes, adrenergic neuron-derived exosomes, gabaergic neuron-derived exosomes, opipoidergic neuron-derived exosomes, cancer-derived exosomes, bone marrow-derived exosomes, lymph node-derived exosomes, prostate-derived exosomes, lung-derived exosomes, liver-derived exosomes, pancreas-derived exosomes, ovary-derived exosomes, gastrointestinal-derived exosomes, kidney and urinary tract-derived exosomes, skin-derived exosomes, bone-derived exosomes, muscle-derived exosomes, peripheral nerve-derived exosomes, adipose tissue-derived exosomes, connective tissue-derived exosomes, male or female organ-derived exosomes, spleen-derived exosomes, endocrine-derived exosomes, and vascular-derived exosomes.

In other embodiments, the present invention provides saturation enzyme-linked immunosorbent assays for detecting exosomes and exosomal biomarkers. In some embodiments, the assay comprises incubating a sample comprising exosomes on a solid support comprising immobilized antibodies, wherein the immobilized antibodies on the solid support are generally saturated by the exosomes in the sample. In certain embodiments, substantially all of the immobilized antibodies are bound to a target antigen from the sample. In certain aspects of the present embodiment, the method further comprises, lysing or permeabilizing the exosomes while maintaining contact between the exosome membrane and the solid support, and detecting cargo or cytosolic biomarkers or inner-membrane bound biomarkers from the captured exosomes. In some embodiments, the cargo or cytosolic biomarker or inner-membrane bound biomarker is selected from the group consisting of a monomer and/or oligomer tau, phosphorylated tau, a synaptic protein, a cytoskeletal protein, and a membrane receptor associated protein and/or kinase. In some embodiments, the synaptic protein is synaptophysin, synaptotagmin, synaptopodin, or SNAP25. In other embodiments, the cytoskeletal protein is a neurofilament. In some embodiments, the cargo or cytosolic biomarker is selected from the group consisting of neurotransmitters and their metabolites, various enzymes including neurotransmitter synthesis and degradation, synaptic proteins, cytoskeletons, cytokines, and chemokines. In some embodiments, the cargo or cytosolic biomarker is α-synuclein, β-amyloid, tau or phosphorylated tau. In other embodiments, the neurotransmitters are selected from the group consisting of acetylcholine, epinephrine, norepinephrine, dopamine, serotonin, histamine, glutamine, gamma-Aminobutyric acid (GABA), N-Methyl-D-aspartate (NMDA), and opioids. In other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. In certain embodiments, the solid support is a plate, a non-magnetic bead, a magnetic bead, a filter, a slide, a wafer, a rod, a particle, a strand, a disc, a membrane, or a surface of a tube, channel, column, flow cell device, or microfluidic device. The solid support can comprise, for example, glass, quartz, silicon, metal, ceramic, plastic, nylon, polyacrylamide, a hydrogel, or a resin. In certain embodiments, the solid support comprises more than one type of capture agent associated therewith, for example, at least 2, 3, 4, 5, 6, 7, 8, 9, 10, 15, 20, or more different capture agents that selectively bind to the exosomes or inner membrane-bound or cargo or cytosolic exosomal biomarkers. In certain embodiments, detection of the inner membrane-bound, cargo or cytosolic biomarkers from the exosomes captured on the solid support comprises using more than one type of detection agent, for example, at least 2, 3, 4, 5, 6, 7, 8, 9, 10, 15, 20, or more different detection agents that selectively bind to different inner membrane-bound, cargo or cytosolic biomarkers from the exosomes. In certain embodiments, the capture agents comprise antibodies, antibody fragments, antibody mimetics, or aptamers that specifically bind to the exosomes. In other embodiments, the detection agents comprise antibodies, antibody fragments, antibody mimetics, or aptamers that specifically bind to the inner membrane-bound, cargo or cytosolic biomarkers from the exosomes. Capture agents and detection agents may comprise monoclonal antibodies, polyclonal antibodies, chimeric antibodies, nanobodies, recombinant fragments of antibodies, Fab fragments, Fab′ fragments, F(ab′)₂ fragments, Fᵥ fragments, or scFᵥ fragments. In certain embodiments, the detection agent further comprises a detectable label, for example, a fluorescent, chemiluminescent, electrochemiluminescent, bioluminescent, isotopic, or radioactive label. In certain embodiments, detecting an inner membrane-bound, cargo or cytosolic biomarker from an exosome comprises performing an immunoassay, such as an enzyme-linked immunosorbent assay (ELISA), an immunofluorescent assay (IFA), an immune-polymerase chain reaction assay, an electro-chemiluminescence immunoassay (ECLIA), or a radioimmunoassay (RIA). The biological sample comprising exosomes may be from a subject or a cell culture supernatant. In certain embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In certain embodiments, a biological sample is obtained from a subject who has been diagnosed or is suspected of having a neurological disorder, such as Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, or Parkinson's disease. In some embodiments, a biological sample is obtained from a subject who has been diagnosed or is suspected of having cancer, such as breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors. In some embodiments, a biological sample is obtained from a subject who has been diagnosed or is suspected of having an immunological disorder. In other embodiments, a biological sample is obtained from a subject who has been diagnosed or is suspected of having a placental disease. In some embodiments, a biological sample is obtained from a subject who has been diagnosed or is suspected of having a hematological disorder, a kidney disease, a gastrointestinal disease, a liver disease, or a musculoskeletal disease. In other embodiments, the subject is selected from the group consisting of a human, a monkey, a dog, a pig, a bovine, a rabbit, a guinea pig, and a rodent. In some embodiments, the methods further comprise diagnosing the subject with a disease or disorder. In other embodiments, the methods further comprise treating the subject for a disease or disorder if the subject is diagnosed as having the disease or disorder.

In certain embodiments, the biomarker is an inner membrane-bound protein or an adsorbed protein on the exosome. In other embodiments, the biomarker may be an exosome surface marker (e.g., CD81, CD63, CD171), a neuron-specific protein (e.g., synaptosome associated protein 25 (SNAP25), neurogranin (NRGN), tau, phosphorylated tau, αβ-42, and synaptophysin), an astrocyte-specific protein (e.g., glial fibrillary acidic protein (GFAP) and excitatory amino acid transporter 1 (EAAT1)), a microglia-specific protein (CD11b), an oligodendrocyte-specific protein (e.g., myelin basic protein (MBP)), an oligodendrocyte myelin glycoprotein (OMG), or a chemokine (CX3CL1) or cytokine (IL1b, IL34, FasL, or IL12B). In another embodiment, the biomarker is a metabolite. In other embodiments, the biomarker is dopamine transporter (DAT). In another embodiment, the method further comprises detecting a cytosolic protein (e.g., glyceraldehyde-3-phosphate dehydrogenase (GAPDH), alpha-synuclein (SNCA), cathepsin D (CTSD), AchE, LAMP1, REST, SYT, GYS, HSP70, BACE, SYMPO, NEFL, caspase, ubiquitin, PSEN1, GSK, PLAP, CSH1, PSG1, or FasL) from the exosomes captured on the solid support. In another embodiment, the method further comprises detecting one or more biomarkers from the exosomes captured on the solid support. In some embodiments, the biomarker is selected from the group consisting of CD81, acetylcholinesterase (AchE), Lysosomal Associated Membrane Protein 1 (LAMP1), CTSD, RE1 Silencing Transcription Factor (REST), synaptotagmin (SYT), NGRN, monocyte chemotactic protein-1 (CCL2), IL34, glycogen synthase (GYS), (OR), death receptor 6 (DR6), heat shock protein (HSP), IL12beta, alpha-beta (Aβ),beta-secretase (BACE), dopamine receptors (D1 and D2), serotonin receptors (2A, 2C, and 3B), GABA receptors (1-6, 5. B1, B2), glutamate receptors (1 and 2), insulin receptors, tumor necrosis factor receptors superfamily (TRAL, TNF receptor, death receptor 5 and 6), neuropeptide receptors (orexin receptor, opioid receptor KOR), EpCAM, PD-L1, ErbB2, CK19, TCR, CD16, CD28, CD32, CD79a, TREM2, and NCAM.

In some embodiments, the biomarker is a neuron-specific protein, an astrocyte-specific protein, a microglia-specific protein, or an oligodendrocyte-specific protein. In other embodiments, the biomarker is a cytosolic protein, a secretory protein, a receptor protein, or an inner-membrane protein. In some embodiments, the cytosolic protein is selected from the group consisting of GAPDH, CTSD, NRGN, MBP, GFAP, Tau, phosphorylated Tau (e.g., T181), synaptophysin, SNCA, tyrosine hydroxgenase (TH), 4-42, AchE, LAMP1, REST, SYT, GYS, HSP70, BACE, SYMPO, NEFL, caspase, ubiquitin, PSEN1, GSK, PLAP, CSH1, PSG1, or FasL. In other embodiments, the secretory protein is selected from the group consisting of cytokines, growth factors, chemokines, and interleukins. In certain aspects, the cytokine is selected from the group consisting of IL1b, IL34, IL6, IL8, IL16, IL23A, IL32, IL33, CX3CL1, CCL2, CXCL12, TNFalpha, TNFSF10, IL12B, nociceptin, GnRH, FasL and TNFSF13. In other embodiments, the neurotransmitter receptor is selected from the group consisting of dopamine receptors (D1 and D2), serotonin receptors (2A, 2C, and 3B), GABA receptors (1-6, 5. B1, B2), glutamate receptors (1 and 2), insulin receptors, tumor necrosis factor receptors superfamily (TRAL, TNF receptor, death receptor 5 and 6), and neuropeptide receptors (orexin receptor, opioid receptor KOR). In other embodiments, the inner-membrane protein is selected from the group consisting of Tau, phosphorylated Tau (e.g., T181, S396), EpCAM, PD-L1, ErbB2, CK19, TCR, CD16, CD28, CD32, CD79a, TREM2, and NCAM. In some embodiments, the biomarker is selected from the group consisting of neurotransmitters and their metabolites, various enzymes including neurotransmitter synthesis and degradation, synaptic proteins, cytoskeletons, cytokines, and chemokines. In some embodiments, the cargo or cytosolic biomarker is α-synuclein, β-amyloid, tau or phosphorylated tau. In other embodiments, the neurotransmitters are selected from the group consisting of acetylcholine, epinephrine, norepinephrine, dopamine, serotonin, histamine, glutamine, gamma-Aminobutyric acid (GABA), N-Methyl-D-aspartate (NMDA), and opioids. In some embodiments, the biomarker is selected from the group consisting of a monomer and/or oligomer tau, phosphorylated tau, a synaptic protein, a cytoskeletal protein, and a membrane receptor associated protein and/or kinase. In some embodiments, the synaptic protein is synaptophysin, synaptotagmin, synaptopodin, or SNAP25. In other embodiments, the cytoskeletal protein is a neurofilament.

In one aspect, the invention provides a method of diagnosing and treating a disease or a disorder in a subject, the method comprising: a) providing a biological sample comprising exosomes; b) contacting a solid support comprising capture agents associated therewith with the biological sample under conditions wherein the capture agents selectively bind to the exosomes, thereby capturing said exosomes on the solid support; c) lysing or permeabilizing the exosomes while maintaining contact between the exosome membrane and the capture agents; d) detecting inner membrane-bound biomarkers from the exosomes; e) diagnosing the subject with the disease or disorder by comparing the level of the biomarker to a control level of the biomarker; and f) treating the subject for the disease or disorder if the subject is diagnosed as having the disease or disorder. In some embodiments, the inner membrane-bound biomarker is selected from the group consisting of a monomer and/or oligomer tau, phosphorylated tau, a synaptic protein, a cytoskeletal protein, and a membrane receptor associated protein and/or kinase. In some embodiments, the synaptic protein is synaptophysin, synaptotagmin, synaptopodin, or SNAP25. In other embodiments, the cytoskeletal protein is a neurofilament. In other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. In certain embodiments, the solid support is a plate, a non-magnetic bead, a magnetic bead, a filter, a slide, a wafer, a rod, a particle, a strand, a disc, a membrane, or a surface of a tube, channel, column, flow cell device, or microfluidic device. The solid support can comprise, for example, glass, quartz, silicon, metal, ceramic, plastic, nylon, polyacrylamide, a hydrogel, or a resin. In certain embodiments, the solid support comprises more than one type of capture agent associated therewith, for example, at least 2, 3, 4, 5, 6, 7, 8, 9, 10, 15, 20, or more different capture agents that selectively bind to the exosomes or inner membrane-bound exosomal biomarkers. In certain embodiments, detection of the inner membrane-bound biomarkers from the exosomes captured on the solid support comprises using more than one type of detection agent, for example, at least 2, 3, 4, 5, 6, 7, 8, 9, 10, 15, 20, or more different detection agents that selectively bind to different inner membrane-bound biomarkers from the exosomes. In certain embodiments, the capture agents comprise antibodies, antibody fragments, antibody mimetics, or aptamers that specifically bind to the exosomes. In other embodiments, the detection agents comprise antibodies, antibody fragments, antibody mimetics, or aptamers that specifically bind to the inner membrane-bound biomarkers from the exosomes. Capture agents and detection agents may comprise monoclonal antibodies, polyclonal antibodies, chimeric antibodies, nobodies, recombinant fragments of antibodies, Fab fragments, Fab′ fragments, F(ab′)₂ fragments, Fᵥ fragments, or scFᵥ fragments. In certain embodiments, the detection agent further comprises a detectable label, for example, a fluorescent, chemiluminescent, electrochemiluminescent, bioluminescent, isotopic, or radioactive label. In certain embodiments, detecting an inner membrane-bound biomarker from an exosome comprises performing an immunoassay, such as an enzyme-linked immunosorbent assay (ELISA), an immunofluorescent assay (IFA), an immune-polymerase chain reaction assay, an electro-chemiluminescence immunoassay (ECLIA), or a radioimmunoassay (RIA). The biological sample comprising exosomes may be from a subject or a cell culture supernatant. In certain embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In certain embodiments, a biological sample is obtained from a subject who has been diagnosed or is suspected of having a neurological disorder, such as Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, or Parkinson's disease. In some embodiments, a biological sample is obtained from a subject who has been diagnosed or is suspected of having cancer, such as breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors. In some embodiments, a biological sample is obtained from a subject who has been diagnosed or is suspected of having an immunological disorder. In other embodiments, a biological sample is obtained from a subject who has been diagnosed or is suspected of having a placental disease. In some embodiments, a biological sample is obtained from a subject who has been diagnosed or is suspected of having a hematological disorder, a kidney disease, a gastrointestinal disease, a liver disease, or a musculoskeletal disease. In other embodiments, the subject is selected from the group consisting of a human, a monkey, a dog, a pig, a bovine, a rabbit, a guinea pig, and a rodent. In some embodiments, the methods further comprise diagnosing the subject with a disease or disorder. In other embodiments, the methods further comprise treating the subject for a disease or disorder if the subject is diagnosed as having the disease or disorder.

In one aspect, the invention provides a method of diagnosing and treating a disease or a disorder in a subject, the method comprising: a) providing a biological sample comprising exosomes; b) contacting a solid support comprising capture agents associated therewith with the biological sample under conditions wherein the capture agents selectively bind to the exosomes, thereby capturing said exosomes on the solid support; c) lysing or permeabilizing the exosomes while maintaining contact between the exosome membrane and the capture agents; d) detecting cargo or cytosolic biomarkers from the exosomes; e) diagnosing the subject with the disease or disorder by comparing the level of the biomarker to a control level of the biomarker; and f) treating the subject for the disease or disorder if the subject is diagnosed as having the disease or disorder. In some embodiments, the cargo or cytosolic biomarker is selected from the group consisting of neurotransmitters and their metabolites, various enzymes including neurotransmitter synthesis and degradation, synaptic proteins, cytoskeletons, cytokines, and chemokines. In some embodiments, the cargo or cytosolic biomarker is α-synuclein, β-amyloid, tau or phosphorylated tau. In other embodiments, the neurotransmitters are selected from the group consisting of acetylcholine, epinephrine, norepinephrine, dopamine, serotonin, histamine, glutamine, gamma-Aminobutyric acid (GABA), N-Methyl-D-aspartate (NMDA), and opioids. In other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. In certain embodiments, the solid support is a plate, a non-magnetic bead, a magnetic bead, a filter, a slide, a wafer, a rod, a particle, a strand, a disc, a membrane, or a surface of a tube, channel, column, flow cell device, or microfluidic device. The solid support can comprise, for example, glass, quartz, silicon, metal, ceramic, plastic, nylon, polyacrylamide, a hydrogel, or a resin. In certain embodiments, the solid support comprises more than one type of capture agent associated therewith, for example, at least 2, 3, 4, 5, 6, 7, 8, 9, 10, 15, 20, or more different capture agents that selectively bind to the exosomes or cargo or cytosolic exosomal biomarkers. In certain embodiments, detection of the cargo or cytosolic biomarkers from the exosomes captured on the solid support comprises using more than one type of detection agent, for example, at least 2, 3, 4, 5, 6, 7, 8, 9, 10, 15, 20, or more different detection agents that selectively bind to different cargo or cytosolic biomarkers from the exosomes. In certain embodiments, the capture agents comprise antibodies, antibody fragments, antibody mimetics, or aptamers that specifically bind to the exosomes. In other embodiments, the detection agents comprise antibodies, antibody fragments, antibody mimetics, or aptamers that specifically bind to the cargo or cytosolic biomarkers from the exosomes. Capture agents and detection agents may comprise monoclonal antibodies, polyclonal antibodies, chimeric antibodies, nanobodies, recombinant fragments of antibodies, Fab fragments, Fab′ fragments, F(ab′)₂ fragments, Fᵥ fragments, or scFᵥ fragments. In certain embodiments, the detection agent further comprises a detectable label, for example, a fluorescent, chemiluminescent, electrochemiluminescent, bioluminescent, isotopic, or radioactive label. In certain embodiments, detecting a cargo or cytosolic biomarker from an exosome comprises performing an immunoassay, such as an enzyme-linked immunosorbent assay (ELISA), an immunofluorescent assay (IFA), an immune-polymerase chain reaction assay, an electro-chemiluminescence immunoassay (ECLIA), or a radioimmunoassay (RIA). The biological sample comprising exosomes may be from a subject or a cell culture supernatant. In certain embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In certain embodiments, a biological sample is obtained from a subject who has been diagnosed or is suspected of having a neurological disorder, such as Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, or Parkinson's disease. In some embodiments, a biological sample is obtained from a subject who has been diagnosed or is suspected of having cancer, such as breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors. In some embodiments, a biological sample is obtained from a subject who has been diagnosed or is suspected of having an immunological disorder. In other embodiments, a biological sample is obtained from a subject who has been diagnosed or is suspected of having a placental disease. In some embodiments, a biological sample is obtained from a subject who has been diagnosed or is suspected of having a hematological disorder, a kidney disease, a gastrointestinal disease, a liver disease, or a musculoskeletal disease. In other embodiments, the subject is selected from the group consisting of a human, a monkey, a dog, a pig, a bovine, a rabbit, a guinea pig, and a rodent. In some embodiments, the methods further comprise diagnosing the subject with a disease or disorder. In other embodiments, the methods further comprise treating the subject for a disease or disorder if the subject is diagnosed as having the disease or disorder.

In one aspect, the invention provides a method of diagnosing and treating a disease or a disorder in a subject, the method comprising: a) providing a biological sample comprising exosomes; b) contacting a solid support comprising capture agents associated therewith with the biological sample under conditions wherein the capture agents selectively bind to the exosomes, thereby capturing said exosomes on the solid support; c) lysing or permeabilizing the exosomes while maintaining contact between the exosome membrane and the capture agents; d) detecting miRNA from the exosomes; e) diagnosing the subject with the disease or disorder by comparing the level of the biomarker to a control level of the biomarker; and f) treating the subject for the disease or disorder if the subject is diagnosed as having the disease or disorder. In other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. In certain embodiments, the solid support is a plate, a non-magnetic bead, a magnetic bead, a filter, a slide, a wafer, a rod, a particle, a strand, a disc, a membrane, or a surface of a tube, channel, column, flow cell device, or microfluidic device. The solid support can comprise, for example, glass, quartz, silicon, metal, ceramic, plastic, nylon, polyacrylamide, a hydrogel, or a resin. In certain embodiments, the solid support comprises more than one type of capture agent associated therewith, for example, at least 2, 3, 4, 5, 6, 7, 8, 9, 10, 15, 20, or more different capture agents that selectively bind to the exosomes or exosomal miRNA. In some embodiments, the methods further comprise incubation of exosomes with Trizol. In certain embodiments, detection of the miRNA from the exosomes captured on the solid support comprises using more than one type of detection agent, for example, at least 2, 3, 4, 5, 6, 7, 8, 9, 10, 15, 20, or more different detection agents that selectively bind to different miRNA from the exosomes. In certain embodiments, the capture agents comprise antibodies, antibody fragments, antibody mimetics, or aptamers that specifically bind to the exosomes. In other embodiments, the detection agents comprise antibodies, antibody fragments, antibody mimetics, or aptamers that specifically bind to the miRNA from the exosomes. In some embodiments, the method further comprises isolating miRNA from the sample by ethanol extraction, magnetic bead separation, or spin column extraction. In some embodiments, the method further comprises determining the level of the miRNA comprising amplifying the miRNA, sequencing the RNA, hybridization, or a gene chip. Capture agents and detection agents may comprise monoclonal antibodies, polyclonal antibodies, chimeric antibodies, nanobodies, recombinant fragments of antibodies, Fab fragments, Fab′ fragments, F(ab′)₂ fragments, Fᵥ fragments, or scFᵥ fragments. In certain embodiments, the detection agent further comprises a detectable label, for example, a fluorescent, chemiluminescent, electrochemiluminescent, bioluminescent, isotopic, or radioactive label. In certain embodiments, detecting miRNA from an exosome comprises performing an immunoassay, such as an enzyme-linked immunosorbent assay (ELISA), an immunofluorescent assay (IFA), an immune-polymerase chain reaction assay, an electro-chemiluminescence immunoassay (ECLIA), or a radioimmunoassay (RIA). The biological sample comprising exosomes may be from a subject or a cell culture supernatant. In certain embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In certain embodiments, a biological sample is obtained from a subject who has been diagnosed or is suspected of having a neurological disorder, such as Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, or Parkinson's disease. In some embodiments, a biological sample is obtained from a subject who has been diagnosed or is suspected of having cancer, such as breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors. In some embodiments, a biological sample is obtained from a subject who has been diagnosed or is suspected of having an immunological disorder. In other embodiments, a biological sample is obtained from a subject who has been diagnosed or is suspected of having a placental disease. In some embodiments, a biological sample is obtained from a subject who has been diagnosed or is suspected of having a hematological disorder, a kidney disease, a gastrointestinal disease, a liver disease, or a musculoskeletal disease. In other embodiments, the subject is selected from the group consisting of a human, a monkey, a dog, a pig, a bovine, a rabbit, a guinea pig, and a rodent. In some embodiments, the methods further comprise diagnosing the subject with a disease or disorder. In other embodiments, the methods further comprise treating the subject for a disease or disorder if the subject is diagnosed as having the disease or disorder

In one aspect, the invention provides a method of diagnosing and treating a disease or a disorder in a subject, the method comprising: incubating a sample comprising exosomes on a solid support comprising immobilized antibodies, wherein the immobilized antibodies on the solid support are generally saturated by the exosomes in the sample; lysing or permeabilizing the exosomes while maintaining contact between the exosome membrane and the solid support; detecting cargo or cytosolic biomarkers or inner-membrane bound biomarkers from the exosomes; and diagnosing the subject with the disease or disorder by comparing the level of the biomarker to a control level of the biomarker; and treating the subject for the disease or disorder if the subject is diagnosed as having the disease or disorder. In certain embodiments, substantially all of the immobilized antibodies are bound to a target antigen from the sample. In some embodiments, the inner membrane-bound biomarker is selected from the group consisting of a monomer and/or oligomer tau, phosphorylated tau, a synaptic protein, a cytoskeletal protein, and a membrane receptor associated protein and/or kinase. In some embodiments, the synaptic protein is synaptophysin, synaptotagmin, synaptopodin, or SNAP25. In other embodiments, the cytoskeletal protein is a neurofilament. In other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. In some embodiments, the cargo or cytosolic biomarker is selected from the group consisting of neurotransmitters and their metabolites, various enzymes including neurotransmitter synthesis and degradation, synaptic proteins, cytoskeletons, cytokines, and chemokines. In some embodiments, the cargo or cytosolic biomarker is α-synuclein, β-amyloid, tau or phosphorylated tau. In other embodiments, the neurotransmitters are selected from the group consisting of acetylcholine, epinephrine, norepinephrine, dopamine, serotonin, histamine, glutamine, gamma-Aminobutyric acid (GABA), N-Methyl-D-aspartate (NMDA), and opioids. In certain embodiments, the solid support is a plate, a non-magnetic bead, a magnetic bead, a filter, a slide, a wafer, a rod, a particle, a strand, a disc, a membrane, or a surface of a tube, channel, column, flow cell device, or microfluidic device. The solid support can comprise, for example, glass, quartz, silicon, metal, ceramic, plastic, nylon, polyacrylamide, a hydrogel, or a resin. In certain embodiments, the solid support comprises more than one type of capture agent associated therewith, for example, at least 2, 3, 4, 5, 6, 7, 8, 9, 10, 15, 20, or more different capture agents that selectively bind to the exosomes or inner membrane-bound or cargo or cytosolic exosomal biomarkers. In certain embodiments, detection of the inner membrane-bound, cargo or cytosolic biomarkers from the exosomes captured on the solid support comprises using more than one type of detection agent, for example, at least 2, 3, 4, 5, 6, 7, 8, 9, 10, 15, 20, or more different detection agents that selectively bind to different inner membrane-bound, cargo or cytosolic biomarkers from the exosomes. In certain embodiments, the capture agents comprise antibodies, antibody fragments, antibody mimetics, or aptamers that specifically bind to the exosomes. In other embodiments, the detection agents comprise antibodies, antibody fragments, antibody mimetics, or aptamers that specifically bind to the inner membrane-bound, cargo or cytosolic biomarkers from the exosomes. Capture agents and detection agents may comprise monoclonal antibodies, polyclonal antibodies, chimeric antibodies, nanobodies, recombinant fragments of antibodies, Fab fragments, Fab′ fragments, F(ab′)₂ fragments, Fᵥ fragments, or scFᵥ fragments. In certain embodiments, the detection agent further comprises a detectable label, for example, a fluorescent, chemiluminescent, electrochemiluminescent, bioluminescent, isotopic, or radioactive label. In certain embodiments, detecting an inner membrane-bound, cargo or cytosolic biomarker from an exosome comprises performing an immunoassay, such as an enzyme-linked immunosorbent assay (ELISA), an immunofluorescent assay (IFA), an immune-polymerase chain reaction assay, an electro-chemiluminescence immunoassay (ECLIA), or a radioimmunoassay (RIA). The biological sample comprising exosomes may be from a subject or a cell culture supernatant. In certain embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In certain embodiments, a biological sample is obtained from a subject who has been diagnosed or is suspected of having a neurological disorder, such as Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, or Parkinson's disease. In some embodiments, a biological sample is obtained from a subject who has been diagnosed or is suspected of having cancer, such as breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors. In some embodiments, a biological sample is obtained from a subject who has been diagnosed or is suspected of having an immunological disorder. In other embodiments, a biological sample is obtained from a subject who has been diagnosed or is suspected of having a placental disease. In some embodiments, a biological sample is obtained from a subject who has been diagnosed or is suspected of having a hematological disorder, a kidney disease, a gastrointestinal disease, a liver disease, or a musculoskeletal disease. In other embodiments, the subject is selected from the group consisting of a human, a monkey, a dog, a pig, a bovine, a rabbit, a guinea pig, and a rodent. In some embodiments, the methods further comprise diagnosing the subject with a disease or disorder. In other embodiments, the methods further comprise treating the subject for a disease or disorder if the subject is diagnosed as having the disease or disorder.

In some embodiments, the level of one or more biomarkers on exosomes in the biological sample is compared to the level of one or more biomarkers in a control sample, wherein the level of the one or more biomarkers of the biological sample is elevated compared to the control sample. In some embodiments, the level of the one or more biomarkers in the biological sample is compared to the level of one or more biomarkers in a control sample, wherein the level of the one or more biomarkers of the biological sample is decreased compared to the control sample. In some embodiments, the biomarker level determines the disease or disorder status of the subject with at least 40%, 50%, 60%, 70%, 75%, 80%, 85%, 90%, 95%, 99%, or 100% specificity. In some embodiments, the disease or disorder is a neurological disorder, immunological disorder, placental disease, cancer, hematological disorder, kidney disease, gastrointestinal disease, liver disease, or musculoskeletal disease. In some embodiments, the neurological disorder is selected from the group consisting of: Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy and Parkinson's disease. In other embodiments, the biological sample is selected from the group consisting of whole blood, serum, plasma, urine, interstitial fluid, peritoneal fluid, cervical swab, tears, saliva, buccal swab, skin, brain tissue, and cerebrospinal fluid. In some embodiments, the cancer is breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors.

These and other embodiments of the present invention will readily occur to those of skill in the art in light of the disclosure herein, and all such embodiments are specifically contemplated.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description. The invention is capable of other embodiments and of being practiced or of being carried out in various ways. Also, the phraseology and terminology used herein is for the purpose of description and should not be regarded as limiting.

### BRIEF DESCRIPTION OF THE DRAWINGS

FIG. 1 sets forth data showing effects of lysis buffer on exosome binding to anti-DAT-immobilized antibody ELISA plate.

FIG. 2 sets forth data showing detection of inner membrane-bound biomarkers from exosomes.

FIGS. 3A-3E set forth data showing detection of inner membrane-bound biomarkers from exosomes in plasma samples from patients with neurological disease compared to controls.

FIG. 4 sets forth data showing exosome binding to anti-SNAP-immobilized solid supports.

FIG. 5 sets forth data showing exosome binding to anti-SNAP-immobilized solid supports.

FIG. 6 sets forth data showing cytosolic SNCA levels from DAT+ exosomes.

FIGS. 7A-7C set forth data showing exosome capture and cytosolic protein detection of GAPDH, total tau and p-tau T181 by ELISA.

FIGS. 8A-8D set forth data showing Saturation ELISA to detect exosomal levels of SNCA.

FIGS. 9A-9D set forth data showing Saturation ELISA to detect exosomal levels of tyrosine hydroxgenase.

FIGS. 10A-10D set forth data showing Saturation ELISA to quantify exosome membranes on anti-DAT plates.

FIGS. 11A-11D set forth data showing Saturation ELISA to detect exosomal levels of SNCA in SNAP25+, EAAT1+, and DAT+ exosomes.

FIG. 12 sets forth data showing current limitations of exosome isolation and preparation using standard ELISA.

FIG. 13. sets forth data showing exosome core isolation according to the methods of the present invention.

FIGS. 14A-14B set forth data showing exosome core isolation according to the methods of the present invention.

FIGS. 15A-15B set forth data showing exosome core isolation according to the methods of the present invention.

FIGS. 16A-16D set forth data showing detection of biomarkers from isolated exosome core.

FIG. 17 sets forth data showing detection of biomarkers in isolated exosome core and exosome membrane.

FIG. 18 sets forth data showing total protein concentrations in exosome membrane and exosome core.

FIGS. 19A-19C set forth data showing heavy metal content in exosome membrane and exosome core.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments, a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The present invention relates, in part, to the development of an efficient method for detecting and quantitating exosomes and exosomal biomarkers. In some embodiments, the invention provides a method for selectively capturing exosomes on a solid support, lysing or permeabilizing the exosomes while maintaining contact between the exosome membrane and the solid support, and detecting inner membrane-bound biomarkers from the captured exosomes. In other embodiments, the invention provides a method for selectively capturing exosomes on a solid support, lysing or permeabilizing the exosomes while maintaining contact between the exosome membrane and the solid support, and detecting cargo or cytosolic biomarkers from the captured exosomes. In yet other embodiments, the invention provides a method for selectively capturing exosomes on a solid support, lysing or permeabilizing the exosomes while maintaining contact between the exosome membrane and the solid support, and detecting miRNA from the captured exosomes. In still other embodiments, the invention provides saturation enzyme-linked immunosorbent assays for detecting exosomes and exosomal biomarkers. In certain embodiments, the assay comprises incubating a sample comprising exosomes on a solid support comprising immobilized antibodies, wherein the immobilized antibodies on the solid support are generally saturated by the exosomes in the sample. In certain embodiments, substantially all of the immobilized antibodies are bound to a target antigen from the sample. In certain aspects of the present embodiment, the method further comprises, lysing or permeabilizing the exosomes while maintaining contact between the exosome membrane and the solid support, and detecting cargo biomarkers or inner-membrane bound biomarkers from the captured exosomes.

Additionally, the present invention provides compositions for use in the methods described herein. Such compositions may include a solid support, capture agents which specifically bind to a biomarker (e.g., an inner-membrane bound protein or cytosolic protein) from exosomes on the solid support, detection agents that specifically bind to biomarkers from exosomes, reagents for performing immunoassays, and other reagents for performing the methods described herein.

The present invention further provides kits for detecting biomarkers from exosomes. The kits may comprise a solid support, one or more capture agents which specifically bind and capture exosomes on the solid support, one or more detection agents which specifically bind a biomarker from exosomes, and optionally, immunoassay reagents and other reagents for performing the methods described herein, one or more containers for collecting and or holding the biological sample, and instructions for using the kits.

The present invention further relates to the discovery that exosomal biomarkers can be assayed to identify subjects having or likely to develop neurological disorders, including, for example, Alzheimer's disease (AD), multiple sclerosis (MS), and frontotemporal dementia (FTD).

The present invention is based, in part, on the discovery of unexpected decreases or increases in certain biomarkers in exosomes present in the circulation of subjects having neurological disease (e.g., Alzheimer's disease). The present invention demonstrates that exosomal levels of these biomarkers may be assayed to diagnose a neurological disorder in a subject having a neurological disease. The present invention further shows that measurement of certain biomarkers in exosomes from a subject may be used to predict the subsequent development of a neurological disease (e.g., identify a subject at risk of developing a neurological disorder).

The section headings are used herein for organizational purposes only, and are not to be construed as in any way limiting the subject matter described herein.

### Biological Sample

A biological sample comprising exosomes may be obtained from a subject. The biological sample obtained from the subject is typically blood, but can be any sample from bodily fluids, tissue or cells comprising the exosomes to be analyzed. The biological sample may include, but is not limited to, whole blood, serum, plasma, urine, interstitial fluid, peritoneal fluid, cerebrospinal fluid, a cervical swab, tears, saliva, a buccal swab, skin, organs, and biopsies. Alternatively, exosomes can be obtained from cultured cells by collection of secreted exosomes from the surrounding culture media.

In some embodiments, the biological sample of the invention is obtained from blood. In some embodiments, about 1-10 mL of blood is drawn from a subject. In other embodiments, about 10-50 mL of blood is drawn from a subject. Blood can be drawn from any suitable area of the body, including an arm, a leg, or blood accessible through a central venous catheter. In some embodiments, blood is collected following a treatment or activity. For example, blood can be collected following a medical exam. The timing of collection can also be coordinated to increase the number and/or composition of exosomes present in the sample. For example, blood can be collected following exercise or a treatment that induces vascular dilation.

Blood may be combined with various components following collection to preserve or prepare samples for subsequent techniques. For example, in some embodiments, blood is treated with an anticoagulant, a cell fixative, a protease inhibitor, a phosphatase inhibitor, a protein, a DNA, or an RNA preservative following collection. In some embodiments, blood is collected via venipuncture using vacuum collection tubes containing an anticoagulant such as EDTA or heparin. Blood can also be collected using a heparin-coated syringe and hypodermic needle. Blood can also be combined with components that will be useful for cell culture. For example, in some embodiments, blood is combined with cell culture media or supplemented cell culture media (e.g., cytokines).

### Enrichment or Isolation of Exosomes

Samples can be enriched for exosomes through positive selection, negative selection, or a combination of positive and negative selection. In some embodiments, exosomes are directly captured. In other embodiments, blood cells are captured and exosomes are collected from the remaining biological samples. In some embodiments, the exosomes enriched in the biological samples are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes.

Samples can also be enriched for exosomes based on differences in the biochemical properties of exosomes. For example, samples can be enriched for exosomes based on antigen, nucleic acid, metabolic, gene expression, or epigenetic differences. In some of the embodiments based on antigen differences, antibody-conjugated magnetic or paramagnetic beads in magnetic field gradients or fluorescently labeled antibodies with flow cytometry are used. In some of the embodiments based on nucleic acid differences, flow cytometry is used. In some of the embodiments based on metabolic differences, dye uptake/exclusion measured by flow cytometry or another sorting technology is used. In some of the embodiments based on gene expression, cell culture with cytokines is used. Samples can also be enriched for exosomes based on other biochemical properties known in the art. For example, samples can be enriched for exosomes based on pH or motility. Further, in some embodiments, more than one method is used to enrich for exosomes. In other embodiments, samples are enriched for exosomes using antibodies, ligands, or soluble receptors.

In other embodiments, surface markers are used to positively enrich exosomes in the sample. In other embodiments, NCAM, CD171, CD9, CD63, CD81, SNAP25, EAAT1, OMG, neuron-specific enolase, diverse neuron or astrocyte adhesive proteins, microglial CD18/11, or CD3 T cell membrane cell surface markers are used to enrich for exosomes. In some embodiments, cell surface markers that are not found on exosomes populations are used to negatively enrich exosomes by depleting cell populations. Flow cytometry sorting may also be used to further enrich for exosomes using cell surface markers or intracellular or extracellular markers conjugated to fluorescent labels. Intracellular and extracellular markers may include nuclear stains or antibodies against intracellular or extracellular proteins preferentially expressed in exosomes. Cell surface markers may include antibodies against cell surface antigens that are preferentially expressed on exosomes (e.g., NCAM). In some embodiments, the cell surface marker is a neuron-derived exosome surface marker, including, for example, NCAM or CD171. In some embodiments, a monoclonal NCAM, CD9, CD63, CD81, neuron-specific enolase or CD171 antibody is used to enrich or isolate exosomes from the sample. In certain aspects, the NCAM, CD9, CD63, CD81, neuron-specific enolase or CD171 antibody is biotinylated. In this embodiment, biotinylated NCAM or CD171 antibody can form an antibody-exosome complex that can be subsequently isolated using streptavidin-agarose resin or beads. In other embodiments, the NCAM, CD9, CD63, CD81, neuron-specific enolase or CD171 antibody is a monoclonal anti-human NCAM, CD9, CD63, CD81, neuron-specific enolase or CD171 antibody. In other embodiments, the cell surface marker is a neuron-specific protein (e.g., synaptosome associated protein 25 (SNAP25), neurogranin (NRGN), tau, phosphorylated tau, aβ-42, and synaptophysin), an astrocyte-specific protein (e.g., glial fibrillary acidic protein (GFAP) and excitatory amino acid transporter 1 (EAAT1)), a microglia-specific protein (CD11b), an oligodendrocyte-specific protein (e.g., myelin basic protein (MBP), an oligodendrocyte myelin glycoprotein (OMG), a cytosolic protein (e.g., glyceraldehyde-3-phosphate dehydrogenase (GAPDH), alpha-synuclein (SNCA), cathepsin D (CTSD), AchE, LAMP1, REST, SYT, GYS, HSP70, BACE, SYMPO, NEFL, caspase, ubiquitin, PSEN1, GSK, PLAP, CSH1, PSG1, or FasL), or a chemokine (CX3CL1) or cytokine (IL1b, IL34, FasL, or IL12B).

In some embodiments, enriched exosomes from the biological sample are subsequently enriched for a specific type of exosome. For example, the biological sample is enriched for exosomes and then the enriched exosomes are subsequently enriched for neural-derived exosomes. In some embodiments, the biological sample is enriched for individual neural cell sources of exosomes. In certain aspects, the neural cell sources of exosomes are microglia, neurons, or astrocytes.

In other embodiments, exosomes are isolated or enriched from a biological sample by a method comprising: contacting a biological sample with an agent under conditions wherein a exosome present in said biological sample binds to said agent to form a exosome-agent complex; and isolating said exosome from said exosome-agent complex to obtain a sample containing said exosome, wherein the purity of exosomes present in said sample is greater than the purity of exosomes present in said biological sample. In certain embodiments, the agent is an antibody or a lectin. Lectins useful for forming a exosome-lectin complex are described in U.S. Patent Application Publication No. 2012/0077263. In other embodiments, the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. In some embodiments, multiple isolating or enriching steps are performed. In certain aspects of the present embodiment, exosomes are isolated from a blood sample comprising exosomes using a solid support, the captured exosomes are subsequently lysed or permeabilized while maintaining contact between the exosome membrane and the solid support, and exosomal biomarkers are detected from the exosomes.

### Capture and Detection of Exosomes on a Solid Support

In other embodiments, exosomes are separated from other molecules in a biological sample using capture agents immobilized on a solid support. Such capture agents bind selectively to a surface marker (e.g., membrane protein or adsorbed protein) on exosomes such that the capture agent can “capture” exosomes having the surface marker. By “capture” is meant that the target exosome can be separated from other molecules in the sample by virtue of the binding of the capture agent to the surface marker on the exosome. mole

The specificity of the capture agent determines the subset of exosomes from a biological sample that are captured on the solid support. One or more capture agents can be used in combination in order to capture exosomes having different surface markers. For example, the solid support may comprise more than one type of capture agent associated therewith, for example, at least 2, 3, 4, 5, 6, 7, 8, 9, 10, 15, 20, or more different capture agents that selectively bind to different biomarkers on the exosomes.

In some embodiments, the capture agent selectively binds to neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes. For example, a capture agent can be chosen that selectively binds to an exosome surface marker (e.g., CD81) to capture exosomes generally, a neuron-specific protein (e.g., synaptosome associated protein 25 (SNAP25), neurogranin (NRGN), tau, phosphorylated tau, aβ-42, and synaptophysin) to capture neuron-derived exosomes, an astrocyte-specific protein (e.g., glial fibrillary acidic protein (GFAP) and excitatory amino acid transporter 1 (EAAT1) to capture astrocyte-derived exosomes, a microglia-specific protein (CD11b) to capture microglia-derived exosomes, an oligodendrocyte-specific protein (e.g., myelin basic protein (MBP) or oligodendrocyte myelin glycoprotein (OMG)) to capture oligodendrocyte-derived exosomes, a cytosolic protein (e.g., glyceraldehyde-3-phosphate dehydrogenase (GAPDH), alpha-synuclein (SNCA) or cathepsin D (CTSD)), or an a chemokine (CX3CL1) or cytokine (IL1b, IL34, FasL, or IL12B) protein to capture a brain-derived exosome.

Typically, the capture agent is associated with a solid support, either directly or indirectly. Capture agents may be immobilized on the surface of a solid support, such as, but not limited to, a plate, slide, wafer, non-magnetic bead, magnetic bead, rod, particle, strand, disc, membrane, film, or the inner surface of a tube, channel, column, flow cell device, or microfluidic device. A solid support may comprise various materials, including, but not limited to glass, quartz, silicon, metal, ceramic, plastic, nylon, polyacrylamide, agarose, resin, porous polymer monoliths, hydrogels, and composites thereof. Additionally, a substrate may be added to the surface of a solid support to facilitate attachment of a capture agent.

Once captured on the solid support, the exosomes can be lysed or permeabilized while maintaining contact between the exosome membrane and the solid support, and detecting one or more biomarkers from the exosomes using detection agents. Such detection agents bind selectively to biomarkers from the exosomes. In certain embodiments, the detection agent selectively binds to a neuron-specific protein (e.g., synaptosome associated protein 25 (SNAP25), neurogranin (NRGN), aβ-42, tau, phosphorylated tau, and synaptophysin), an astrocyte-specific protein (e.g., glial fibrillary acidic protein (GFAP) and excitatory amino acid transporter 1 (EAAT1)), a microglia-specific protein (CD11b), an oligodendrocyte-specific protein (e.g., myelin basic protein (MBP), an oligodendrocyte myelin glycoprotein (OMG), a cytosolic protein (e.g., glyceraldehyde phosphate dehydrogenase (GAPDH), alpha-synuclein (SNCA), cathepsin D (CTSD), AchE, LAMP1, REST, SYT, GYS, HSP70, BACE, SYMPO, NEFL, caspase, ubiquitin, PSEN1, GSK, PLAP, CSH1, PSG1, or FasL), a chemokine (CX3CL1) or cytokine (IL1b, IL34, FasL, or IL12B), CTSD, GAPDH, CD81, CD63, or CD171, AchE, LAMP1, REST, SYT, CCL2, IL34, GYS, OR, DR6, HSP, IL12beta, A13, or BACE. In certain embodiments, detection of biomarkers on exosomes captured on the solid support comprises using more than one type of detection agent, for example, at least 2, 3, 4, 5, 6, 7, 8, 9, 10, 15, 20, or more different detection agents that selectively bind to different biomarkers on the exosomes.

In some embodiments, the invention provides a method comprising the steps of: a) providing a biological sample comprising exosomes; b) contacting a solid support comprising capture agents associated therewith with the biological sample under conditions wherein the capture agents selectively bind to the exosomes, thereby capturing said exosomes on the solid support; c) lysing or permeabilizing the exosomes while maintaining contact between the exosome and the solid support, and between exosome membrane-bound biomarker and exosome membrane; and d) isolating exosome core or cargo from the solid support. In some embodiments, the methods further comprises detecting cargo and/or cytosolic biomarkers from the exosome core. The isolation of exosome core and the detection of cargo and/or cytosolic biomarkers from the isolated exosome core are disclosed in Example 5.

Capture agents and detection agents may comprise, for example, antibodies, antibody fragments, antibody mimetics, or aptamers that specifically bind to a surface marker (e.g., membrane-bound or adsorbed protein) on an exosome. The phrase “specifically (or selectively) binds” refers to a binding reaction that is determinative of the presence of the surface marker on a exosome in a heterogeneous population of proteins and other biologics. Thus, under designated assay conditions, the specified capture agents or detection agents bind to a particular surface marker on an exosome at least two times the background and do not substantially bind in a significant amount to other proteins present in the sample. Capture agents and detection agents may comprise, for example, antibodies, antibody fragments, antibody mimetics, or aptamers that specifically bind to an inner-membrane bound biomarker on an exosome. Capture agents and detection agents may comprise, for example, antibodies, antibody fragments, antibody mimetics, or aptamers that specifically bind to a cargo or cytosolic biomarker on an exosome.

In certain embodiments, the capture agent or detection agent comprises an antibody that specifically binds to a surface marker on an exosome, an inner-membrane bound biomarker from an exosome, or a cargo or cytosolic biomarker from an exosome. Any type of antibody may be used, including polyclonal and monoclonal antibodies, hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule (i.e., specifically binds to a target surface marker on an exosome).

In other embodiments, the capture agent or detection agent comprises an aptamer that specifically binds to the target surface marker on an exosome, an inner-membrane bound biomarker from an exosome, or a cargo or cytosolic biomarker from an exosome. Any type of aptamer may be used, including a DNA, RNA, xeno-nucleic acid (XNA), or peptide aptamer that specifically binds to the target antibody isotype. Such aptamers can be identified, for example, by screening a combinatorial library. Nucleic acid aptamers (e.g., DNA or RNA aptamers) that bind selectively to a target antibody isotype can be produced by carrying out repeated rounds of in vitro selection or systematic evolution of ligands by exponential enrichment (SELEX). Peptide aptamers that bind to a target antibody isotype may be isolated from a combinatorial library and improved by directed mutation or repeated rounds of mutagenesis and selection. For a description of methods of producing aptamers, see, e.g., Aptamers: Tools for Nanotherapy and Molecular Imaging (R. N. Veedu ed., Pan Stanford, 2016), Nucleic Acid and Peptide Aptamers: Methods and Protocols (Methods in Molecular Biology, G. Mayer ed., Humana Press, 2009), Nucleic Acid Aptamers: Selection, Characterization, and Application (Methods in Molecular Biology, G. Mayer ed., Humana Press, 2016), Aptamers Selected by Cell-SELEX for Theranostics (W. Tan, X. Fang eds., Springer, 2015), Cox et al. (2001) Bioorg. Med. Chem. 9(10):2525-2531; Cox et al. (2002) Nucleic Acids Res. 30(20): e108, Kenan et al. (1999) Methods Mol Biol. 118:217-231; Platella et al. (2016) Biochim. Biophys. Acta November 16 pii: S0304-4165(16)30447-0, and Lyu et al. (2016) Theranostics 6(9):1440-1452; herein incorporated by reference in their entireties.

In yet other embodiment, the capture agent or detection agent comprises an antibody mimetic. Any type of antibody mimetic may be used, including, but not limited to, affibody molecules (Nygren (2008) FEBS J. 275 (11):2668-2676), affitins (Ebersbach et al. (2007) J. Mol. Biol. 372 (1):172-185), affimers (Johnson et al. (2012) Anal. Chem. 84 (15):6553-6560), affitins (Krehenbrink et al. (2008) J. Mol. Biol. 383 (5):1058-1068), alphabodies (Desmet et al. (2014) Nature Communications 5:5237), anticalins (Skerra (2008) FEBS J. 275 (11):2677-2683), avimers (Silverman et al. (2005) Nat. Biotechnol. 23 (12):1556-1561), darpins (Stumpp et al. (2008) Drug Discov. Today 13 (15-16):695-701), fynomers (Grabulovski et al. (2007) J. Biol. Chem. 282 (5):3196-3204), and monobodies (Koide et al. (2007) Methods Mol. Biol. 352:95-109).

Detection agents may further comprise a detectable label to facilitate detection and/or quantitation of biomarkers from exosomes. Detectable labels include fluorescent, chemiluminescent, electrochemiluminescent, or bioluminescent tags, metals, dyes, radionuclides, and the like, attached to the specific binding agent (e.g., antibody, antibody fragment, antibody mimetic, or aptamer that specifically binds to a membrane-bound or adsorbed biomarker on exosomes).

### Neurological Disorders

The present invention provides methods for diagnosing or prognosing a neurological disorder in a subject, identifying a subject at risk of a neurological disorder, or prescribing a therapeutic regimen or predicting benefit from therapy in a subject having a neurological disorder. In some embodiments, the present invention provides methods for differential diagnosis of a neurological disorder in a subject.

In some embodiments the neurological disorder is selected from the group consisting of: Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia and Parkinson's disease.

In some embodiments, the present invention enables a medical practitioner to diagnose or prognose one or more neurological disorders in a subject. In other embodiments, the present invention enables a medical practitioner to rule out or eliminate one or more neurological diseases as a diagnostic possibility. In other embodiments, the methods of the present invention allow a medical practitioner to distinguish some forms of FTD from Alzheimer's disease. In yet other embodiments, the present invention enables a medical practitioner to identify a subject at risk of developing a neurological disorder. In other embodiments, the present invention enables a medical practitioner to predict whether a subject will later develop a neurological disorder. In further embodiments, the present invention enables a medical practitioner to prescribe a therapeutic regimen or predict benefit from therapy in a subject having a neurological disorder.

### Cancer

The present invention provides methods for diagnosing or prognosing cancer in a subject, identifying a subject at risk of developing cancer, or prescribing a therapeutic regimen or predicting benefit from therapy in a subject having cancer. Generally, a cancer is characterized by the uncontrolled growth of abnormal cells anywhere in a body. The abnormal cells may be termed cancer cells, malignant cells, or tumor cells. Cancer is not confined to humans; animals and other living organisms can get cancer.

In some instances, the cancer may be malignant. Alternatively, the cancer may be benign. The cancer may be a recurrent and/or refractory cancer. Most cancers can be classified as a carcinoma, sarcoma, leukemia, lymphoma, myeloma, or a central nervous system cancer.

The cancer may be a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Alternatively, the cancer may be a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. Preferably, the cancer is a prostate cancer. Alternatively, the cancer may be a thyroid cancer, bladder cancer, or pancreatic cancer.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavity (pleura) or lining of the abdomen (peritoneum). The mesothelioma may be due to asbestos exposure. The cancer may be a brain cancer, such as a glioblastoma.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The cancer may be a leukemia. The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic-leukemia.

In some instances, the cancer is a lymphoma. Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

In some embodiments, the present invention enables a medical practitioner to diagnose or prognose one or more cancers in a subject. In other embodiments, the present invention enables a medical practitioner to rule out or eliminate one or more cancers as a diagnostic possibility. In other embodiments, the methods of the present invention allow a medical practitioner to identify the origin of a cancer. In yet other embodiments, the present invention enables a medical practitioner to identify a subject at risk of developing cancer. In other embodiments, the present invention enables a medical practitioner to predict whether a subject will later develop cancer. In further embodiments, the present invention enables a medical practitioner to prescribe a therapeutic regimen or predict benefit from therapy in a subject having cancer. Exemplary biomarkers of the present invention that are useful in cancer diagnosis and prognosis include, but are not limited to, EpCAM, PD-L1, ErbB2, CK19.

### Immunological Disorders

The present invention provides methods for diagnosing or prognosing an immunological disorder in a subject, identifying a subject at risk of developing an immunological disorder, or prescribing a therapeutic regimen or predicting benefit from therapy in a subject having an immunological disorder. Immunological disorders are diseases or conditions caused by a dysfunction of the immune system and include allergy, asthma, autoimmune diseases, autoinflammatory syndromes and immunological deficiency syndromes.

In some embodiments, the present invention enables a medical practitioner to diagnose or prognose one or more immunological disorders in a subject. In other embodiments, the present invention enables a medical practitioner to rule out or eliminate one or more immunological disorders as a diagnostic possibility. In yet other embodiments, the present invention enables a medical practitioner to identify a subject at risk of developing an immunological disorder. In other embodiments, the present invention enables a medical practitioner to predict whether a subject will later develop an immunological disorder. In further embodiments, the present invention enables a medical practitioner to prescribe a therapeutic regimen or predict benefit from therapy in a subject having an immunological disorder. Exemplary biomarkers of the present invention that are useful in immunological disorder diagnosis and prognosis include, but are not limited to, TCR, CD16, CD28, CD32, CD79a, and TREM2.

### Placental Disease and Fetal Assessment

The present invention provides methods for diagnosing or prognosing placental disease in a subject, identifying a subject at risk of developing a placental disease, or prescribing a therapeutic regimen or predicting benefit from therapy in a subject having a placental disease. Generally, a placental disease is any disease, disorder, or pathology of the placenta. The methods and biomarkers of the present invention may also be used for fetal assessment or diagnosis of fetal disorders, such as, for example, fetal alcohol syndrome or fetal genetic abnormalities. Exemplary biomarkers of the present invention that are useful in placental disease or fetal assessment diagnosis and prognosis include, but are not limited to, PLAP, CSH1, and PSG1.

### Biomarkers

Biomarker levels from exosomes are assayed in a biological sample obtained from a subject having or at-risk of having a disease. In some embodiments, biomarker levels from exosomes are assayed in a biological sample obtained from a subject having or at-risk of having a neurological disorder (e.g., Alzheimer's disease). In other embodiments, biomarker levels from exosomes are assayed in a biological sample obtained from a subject having or at-risk of having cancer. In yet other embodiments, biomarker levels from exosomes are assayed in a biological sample obtained from a subject having or at-risk of having an immunological disorder. In still other embodiments, biomarker levels from exosomes are assayed in a biological sample obtained from a subject having or at-risk of having a placental disorder. In some embodiments, one or more biomarkers are selected from the group consisting of a neuron-specific protein (e.g., synaptosome associated protein 25 (SNAP25), neurogranin (NRGN), tau, and synaptophysin), an astrocyte-specific protein (e.g., glial fibrillary acidic protein (GFAP) and excitatory amino acid transporter 1 (EAAT1)), a microglia-specific protein (CD11b), and an oligodendrocyte-specific protein (e.g., myelin basic protein (MBP), an oligodendrocyte myelin glycoprotein (OMG)). In another embodiment, the biomarkers are CD171, phosphorylated tau T181, SNCA, and NRGN. In other embodiments, the biomarkers are acetylcholinesterase (AchE), Lysosomal Associated Membrane Protein 1 (LAMP1), CTSD, RE1 Silencing Transcription Factor (REST), synaptotagmin (SYT), monocyte chemotactic protein-1 (CCL2), IL34, glycogen synthase (GYS), (OR), death receptor 6 (DR6), heat shock protein (HSP), IL12beta, alpha-beta (Aβ), and beta-secretase (BACE). In some embodiments, one or more biomarkers are selected from the group consisting of a cytosolic proteins, secretory proteins, membrane proteins and receptors and their pathological forms, including aggregates and mutated ones. Biomarkers of the present invention include neurotransmitter receptors, such as, for example, dopamine receptors (D1 and D2), serotonin receptors (2A, 2C, and 3B), GABA receptors (1-6, 5. B1, B2), and glutamate receptors (1 and 2). Other receptor biomarkers of the present invention include, insulin receptors, tumor necrosis factor receptors superfamily (TRAL, TNF receptor, death receptor 5 and 6), and neuropeptide receptors (orexin receptor, opioid receptor KOR). Biomarkers of the present invention include membrane proteins, such as, for example, EpCAM, PD-L1, ErbB2, CK19, TCR, CD16, CD28, CD32, CD79a, TREM2, and NCAM. Other known neurological disorder biomarkers may be used in combination with the biomarkers of the present invention. Examples of such biomarkers are provided in US Patent Application Pub. No. 2015/0119278, the contents of which are hereby incorporated by reference.

In some embodiments, the biomarker is a mutated protein, an over- or under expressed protein, a modified protein (e.g., phosphorylated protein, methylated protein, glycosylated protein, acetylated protein, or ubiquitinated protein), an organ/tissue-specific protein (e.g., neurofilaments for neuron, GFAP for astrocytes, surfactant proteins for lung, PSA for prostate), a disease-specific protein (e.g., Tau or Abeta), a protein monomer and/or oligomer, an enzyme, a kinase, a hormone, a growth factor, a transcription factor, a cytokine and/or chemokine, an RNA (e.g., miRNA, mRNA, and/or non-coding RNA), a neurotransmitter (e.g., acetylcholine, dopamine, serotonin, opioid), a small molecule (e.g. drug or nitric oxide), a metabolite, a lipid and/or lipoprotein, a metal, or a foreign body (e.g., bacteria and/or viral origin biomolecules). In some embodiments, the biomarker is Tau monomers and/or oligomers. In other embodiments the biomarker is alpha-synuclein. In yet other embodiments, the biomarker is phosphorylated-Tau or Abeta monomers and/or oligomers. In still other embodiments, the biomarker is TDP-43 or SOD.

One of ordinary skill in the art has several methods and devices available for the detection and analysis of the markers of the instant invention. With regard to polypeptides or proteins on exosomes in patient test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amounts of analytes without the need for a labeled molecule.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, an enzyme-linked immunosorbent assay (ELISA), immunofluorescent assay (IFA), immune-polymerase chain reaction assay, electro-chemiluminescence immunoassay (ECLIA), radioimmunoassay (RIA), competitive binding assay, planar waveguide technology, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the biomarkers from exosomes is also contemplated by the present invention. The antibodies could be immobilized onto a variety of solid supports, such as magnetic or chromatographic matrix particles, the surface of an assay place (such as microtiter wells), pieces of a solid substrate material (such as plastic, nylon, paper), and the like. An assay strip could be prepared by coating the antibody or a plurality of antibodies in an array on solid support. This strip could then be dipped into the test sample to capture exosomes through binding to surface markers, and then processed quickly through washes and detection steps with detection reagents, as described above, to generate a measurable signal, such as a colored spot.

The analysis of a plurality of biomarkers may be carried out separately or simultaneously with one test sample. Several markers from exosomes may be captured and/or detected using a combination of multiple capture agents and/or detection agents in one test for efficient processing of multiple of samples. In addition, one skilled in the art would recognize the value of testing multiple samples (for example, at successive time points) from the same individual. Such testing of serial samples will allow the identification of changes in biomarker levels over time. Increases or decreases in biomarker levels, as well as the absence of change in biomarker levels, would provide useful information about the disease status that includes, but is not limited to identifying the approximate time from onset of the event, the presence and amount of salvageable tissue, the appropriateness of drug therapies, the effectiveness of various therapies, identification of the severity of the event, identification of the disease severity, and identification of the patient's outcome, including risk of future events.

An assay consisting of a combination of the biomarkers referenced in the instant invention may be constructed to provide relevant information related to differential diagnosis. Such a panel may be constructed using 1, 2, 3, 4, 5, 6, 7, 8, 9, 10, 15, 20, or more or individual biomarkers. The analysis of a single biomarker or subsets of biomarkers comprising a larger panel of markers could be carried out methods described within the instant invention to optimize clinical sensitivity or specificity in various clinical settings.

The analysis of exosomal biomarkers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different analytes. Such formats include protein microarrays, or “protein chips” and capillary devices.

Biomarkers of the present invention serve an important role in the early detection and monitoring of diseases and disorders (e.g., Alzheimer's disease). Biomarkers of such disorders are typically substances found in a bodily sample that can be measured. The measured amount can correlate to underlying disorder or disease pathophysiology, presence or absence of a neurological disorder, probability of a neurological disorder in the future. In patients receiving treatment for their condition the measured amount will also correlate with responsiveness to therapy. In some embodiments, a decrease or increase in the level of one or more biomarkers of the present invention is indicative of a neurological disorder. For example, as shown in Example 1 herein, an increase in inner membrane-bound tau levels from exosomes is indicative of Alzheimer's disease. Accordingly, the methods of the present invention are useful for the diagnosis of Alzheimer's disease.

In some embodiments, a biomarker is measured by a method selected from the group consisting of immunohistochemistry, immunocytochemistry, immunofluorescence, immunoprecipitation, electro-chemiluminescence immunoassay, radioimmunoassay, immune-polymerase chain reaction, western blotting, and ELISA.

### Clinical Assay Performance

The methods of the present invention may be used in clinical assays to diagnose or prognose a neurological disorder in a subject, identify a subject at risk of a neurological disorder, and/or for prescribing a therapeutic regimen or predicting benefit from therapy in a subject having a neurological disorder. Clinical assay performance can be assessed by determining the assay's sensitivity, specificity, area under the ROC curve (AUC), accuracy, positive predictive value (PPV), and negative predictive value (NPV). Disclosed herein are assays for diagnosing or prognosing a neurological disorder in a subject, identifying a subject at risk of a neurological disorder, or for prescribing a therapeutic regimen or predicting benefit from therapy in a subject having a neurological disorder.

The clinical performance of the assay may be based on sensitivity. The sensitivity of an assay of the present invention may be at least about 40%, 45%, 50%, 55%, 60%, 65%, 70%, 75%, 80%, 85%, 90%, 95%, 99%, or 100%. The clinical performance of the assay may be based on specificity. The specificity of an assay of the present invention may be at least about 40%, 45%, 50%, 55%, 60%, 65%, 70%, 75%, 80%, 85%, 90%, 95%, 99%, or 100%. The clinical performance of the assay may be based on area under the ROC curve (AUC). The AUC of an assay of the present invention may be at least about 0.5, 0.55, 0.6, 0.65, 0.7, 0.75, 0.8, 0.85, 0.9, or 0.95. The clinical performance of the assay may be based on accuracy. The accuracy of an assay of the present invention may be at least about 40%, 45%, 50%, 55%, 60%, 65%, 70%, 75%, 80%, 85%, 90%, 95%, 99%, or 100%.

### Compositions

Compositions useful in the methods of the present invention include compositions that specifically recognize a biomarker associated with a disease or disorder. Such compositions may include capture agents and/or detection agents that recognize, for example, a neuron-specific protein biomarker, such as synaptosome associated protein 25 (SNAP25), aβ-42, neurogranin (NRGN), tau, phosphorylated tau, and synaptophysin, an astrocyte-specific protein biomarker, such as glial fibrillary acidic protein (GFAP) and excitatory amino acid transporter 1 (EAAT1), an oligodendrocyte-specific protein biomarker, such as myelin basic protein (MBP) and oligodendrocyte myelin glycoprotein (OMG), a microglia-specific protein (CD11b), a cytosolic protein (e.g., glyceraldehyde-3-phosphate dehydrogenase (GAPDH), alpha-synuclein (SNCA), cathepsin D (CTSD), AchE, LAMP1, REST, SYT, GYS, HSP70, BACE, SYMPO, NEFL, caspase, ubiquitin, PSEN1, GSK, PLAP, CSH1, PSG1, or FasL), or a chemokine (CX3CL1) or cytokine (IL1b, IL34, FasL, or IL12B).

In yet other embodiments, the composition is selected from the group consisting of a peptide, a nucleic acid, an antibody, and a small molecule.

In certain embodiments, the present invention relates to compositions that specifically detect a biomarker associated with a disease or disorder. As detailed elsewhere herein, the present invention is based upon the finding that GAPDH, CTSD, NRGN, MBP, GFAP, Tau, phosphorylated Tau, synaptophysin, aβ-42, CX3CL1, IL1b, IL34, CD81, CD63, CD171, SNAP25, EAAT1, SNCA, CD11b, OMG, AchE, LAMP1, REST, SYT, CCL2, IL34, GYS, OR, DR6, HSP, IL12b, A13, and BACE can be used as biomarkers for AD and other neurological disorders. In some embodiments, the compositions of the present invention specifically bind to and detect such biomarkers. For example, a composition may comprise a solid support comprising capture agents associated therewith that selectively bind to CD81, CD63, CD171, SNAP25, EAAT1, CD11b, or OMG. In another example, a composition may comprise detection agents that selectively bind to GAPDH, CTSD, NRGN, MBP, GFAP, Tau, phosphorylated Tau, synaptophysin, aβ-42, SNCA, CX3CL1, IL1b, IL34, OMG, AchE, LAMP1, REST, SYT, CCL2, IL34, GYS, OR, DR6, HSP, IL12b, A13, and/or BACE.

In some embodiments, the composition comprises an antibody, where the antibody specifically binds to a biomarker or exosomes of the invention. The term “antibody” as used herein and further discussed below is intended to include fragments thereof which are also specifically reactive with a biomarker or exosome. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. Antigen-binding portions may also be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), single domain antibodies, bispecific antibodies, chimeric antibodies, humanized antibodies, diabodies and polypeptides that contain at least a portion of an immunoglobulin that is sufficient to confer specific antigen binding to the polypeptide. In certain embodiments, the antibody further comprises a label attached thereto and able to be detected (e.g., the label can be a radioisotope, fluorescent compound, enzyme or enzyme co-factor).

In certain embodiments, an antibody of the invention is a monoclonal antibody, and in certain embodiments, the invention makes available methods for generating novel antibodies that specifically bind the biomarker or the exosome of the invention. For example, a method for generating a monoclonal antibody that specifically binds a biomarker or exosome, may comprise administering to a mouse an amount of an immunogenic composition comprising the biomarker or exosome, or fragment thereof, effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the biomarker or exosome. Once obtained, a hybridoma can be propagated in a cell culture, optionally in culture conditions where the hybridoma-derived cells produce the monoclonal antibody that binds specifically to the biomarker or exosome. The monoclonal antibody may be purified from the cell culture.

The term “specifically reactive with” as used in reference to an antibody is intended to mean, as is generally understood in the art, that the antibody is sufficiently selective between the antigen of interest (e.g., a biomarker or exosome) and other antigens that are not of interest. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

Antibodies can be generated to bind specifically to an epitope of an exosome or a biomarker of the present invention, including, for example, neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes, or neuron-specific proteins selected from the group consisting of synaptosome associated protein 25 (SNAP25), neurogranin (NRGN), tau, phosphorylated tar, and synaptophysin, astrocyte-specific proteins selected from the group consisting of glial fibrillary acidic protein (GFAP) and excitatory amino acid transporter 1 (EAAT1), and oligodendrocyte-specific proteins selected from the group consisting of myelin basic protein (MBP) and oligodendrocyte myelin glycoprotein (OMG), a microglia-specific protein (CD11b), and chemokine (CX3CL1) or cytokine (IL1b, IL34, FasL, or IL12B). In another embodiment, the antibody generated is an anti-CD171 antibody, an anti-synaptosome associated protein 25 (SNAP25) antibody, an anti-neurogranin (NRGN) antibody, an anti-tau antibody, an anti-synaptophysin antibody, and anti-CD63 antibody, an anti-4-42 antibody, an anti-CD81 antibody, an anti-CTD antibody, an anti-GAPDH antibody, an anti-IL1b antibody, an anti-IL34 antibody, an anti-CX3CL1 antibody, an anti-glial fibrillary acidic protein (GFAP) antibody, an anti-excitatory amino acid transporter 1 (EAAT1) antibody, an anti-SNCA antibody, and anti-CD11b antibody, an anti-myelin basic protein (MBP) antibody, an anti-oligodendrocyte myelin glycoprotein (OMG) antibody, an anti-AchE antibody AchE, an anti-LAMP1 antibody LAMP1, an anti-REST antibody REST, an anti-SYT antibody SYT, an anti-CCL2 antibody CCL2, an anti-IL34 antibody IL34, an anti-GYS antibody GYS, an anti-OR antibody OR, an anti-DR6 antibody DR6, an anti-HSP antibody HSP, an anti-IL12b antibody IL12b, an anti-A13 antibody A13, or an anti-BACE antibody BACE.

In addition, the techniques used to screen antibodies in order to identify a desirable antibody may influence the properties of the antibody obtained. A variety of different techniques are available for testing interaction between antibodies and antigens to identify particularly desirable antibodies. Such techniques include ELISAs, surface plasmon resonance binding assays (e.g., the Biacore binding assay, Biacore AB, Uppsala, Sweden), sandwich assays (e.g., the paramagnetic bead system of IGEN International, Inc., Gaithersburg, Md.), western blots, immunoprecipitation assays, immunocytochemistry, and immunohistochemistry.

In some embodiments, the present invention relates to compositions used for treating or preventing a disease or disorder. As detailed elsewhere herein, the present invention is based upon the findings that the levels of CD81, GAPDH, CTSD, NRGN, MBP, GFAP, Tau, phosphorylated Tau (e.g., T181), synaptophysin, CD63, 4-42, SNCA, CX3CL1, IL1b, IL34, AchE, LAMP1, REST, SYT, CCL2, IL34, GYS, OR, DR6, HSP, IL12b, Aβ, and/or BACE are implicated in the pathology of a variety of disease and disorders, such as, for example, Alzheimer's disease.

In some embodiments, biomarkers inside exosomes are analyzed in addition to the inner membrane-bound biomarkers. In certain embodiments, the present invention relates to compositions for lysing or permeabilizing exosomes in biological samples obtained from a subject. Lytic agents useful in the methods of the present invention include: RIPA buffer; Tris-HCl (pH 6.8); glycerol; SDS; 2-mercaptoethanol; Triton-X 100; M-PER Reagent; T-PER solution; TRISOL, and CHAPS. Lytic agents may be incubated with biological samples to disrupt the membrane of the exosomes of the present invention and release exosome cargo (e.g., exosomal proteins) for subsequent analysis.

### Methods of Treatment

The present invention provides methods of treating a disease or disorder in a subject, comprising administering to the subject an effective amount of a composition, wherein the composition increases, decreases, or maintains the level of CD81, GAPDH, CTSD, NRGN, MBP, GFAP, Tau, phosphorylated Tau (e.g., T181), synaptophysin, CD63, aβ-42, SNCA, CX3CL1, IL1b, or IL34 in the subject. In yet other embodiments, the composition prevents increases or decreases in CD81, GAPDH, CTSD, NRGN, MBP, GFAP, Tau, phosphorylated Tau (e.g., T181), synaptophysin, CD63, aβ-42, SNCA, CX3CL1, IL1b, IL34, AchE, LAMP1, REST, SYT, CCL2, IL34, GYS, OR, DR6, HSP, IL12b, A13, or BACE levels. In other embodiments, the present invention provides methods of treating a disease or disorder in a subject, comprising administering to the subject an effective amount of a composition, wherein the composition normalizes the level of CD81, GAPDH, CTSD, NRGN, MBP, GFAP, Tau, phosphorylated Tau (e.g., T181), synaptophysin, CD63, aβ-42, SNCA, CX3CL1, IL1b, IL34, AchE, LAMP1, REST, SYT, CCL2, IL34, GYS, OR, DR6, HSP, IL12b, Aβ, and/or BACE to a reference level.

### Kits

The above-described assay reagents, including a solid support with bound capture agents, detection agents, and optionally reagents for performing immunoassays, such as by ELISA, IFA, immune-polymerase chain reaction assay, ECLIA, or RIA, can be provided in kits, with suitable instructions and other necessary reagents, in order to conduct the assays for detecting biomarkers on exosomes, as described above. The kit will normally contain in separate containers the solid support with bound capture agents, detection agents, control formulations (positive and/or negative), and other reagents that the assay format requires. Instructions (e.g., written, CD-ROM, DVD, Blu-ray, flash drive, digital download, etc.) for carrying out the assay usually will be included in the kit. The kit can also contain, depending on the particular assay used, other packaged reagents and materials (i.e., wash buffers, and the like). Assays, such as those described above, can be conducted using these kits.

In another embodiment, the invention encompasses kits for detecting or monitoring a disease or disorder in a subject. A variety of kits having different components are contemplated by the current invention. Generally speaking, the kit will include the means for quantifying one or more biomarkers in a subject. In another embodiment, the kit will include means for collecting a biological sample, means for quantifying one or more biomarkers in the biological sample, and instructions for use of the kit contents. In certain embodiments, the kit comprises a means for enriching or isolating exosomes in a biological sample. In further aspects, the kit comprises a solid support with bound capture agents for isolating exosomes from a biological sample. In certain aspects, the kit comprises a means for quantifying the amount of a biomarker. In further aspects, the means for quantifying the amount of a biomarker comprises reagents necessary to detect the amount of a biomarker.

In another embodiment, the invention includes a kit for diagnosing or prognosing a disease or disorder in a subject, identifying a subject at risk of a disease or disorder, or prescribing a therapeutic regimen or predicting benefit from therapy in a subject having a disease or disorder, the kit comprising: a) a solid support comprising capture agents associated therewith, wherein at least one capture agent selectively binds to CD171, CD63, CD81, SNAP25, EAAT1, or OMG; and b) one or more detection agents, wherein the one or more detection agents selectively binds to CD81, GAPDH, CTSD, NRGN, MBP, GFAP, Tau, phosphorylated Tau (e.g., T181), CD63, aβ-42, CX3CL1, IL1b, IL34, AchE, LAMP1, REST, SYT, CCL2, IL34, GYS, OR, DR6, HSP, IL12b, A13, or BACE on the surface of the exosomes. In certain embodiments, at least one capture agent or detection agent comprises an antibody, an antibody fragment, an antibody mimetic, or an aptamer that specifically binds to CD171, phosphorylated tau T181, or neurogranin. In certain embodiments, the antibody is selected from the group consisting of a monoclonal antibody, a polyclonal antibody, a chimeric antibody, a nanobody, a recombinant fragment of an antibody, an Fab fragment, an Fab′ fragment, an F(ab′)₂ fragment, an Fᵥ fragment, and an scFᵥ fragment. In another embodiment, the kit comprises an anti-neurogranin antibody, an anti-phosphorylated tau T181 antibody, and an anti-CD171 antibody.

These and other embodiments of the present invention will readily occur to those of ordinary skill in the art in view of the disclosure herein.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications are intended to fall within the scope of the appended claims.

### Example 1: Detection of Inner Membrane-Bound Biomarkers from Exosomes

Inner membrane-bound biomarkers from exosomes were detected as follows. To capture exosomes, four plasma samples were added to duplicate wells on an anti-DAT-immobilized (dopamine transporter) ELISA plate. Prior to reacting with biotinylated anti-CD81 probes, one well for each sample was exposed to a lysis buffer and the other well for the sample was suspended in PBS buffer. Incubation of all wells was continued for 1 hour. Next, biotinylated anti-CD81 antibodies was added to all wells to react with exosomes followed by streptavidin-horseradish peroxidase reaction for chemiluminescent ELISA.

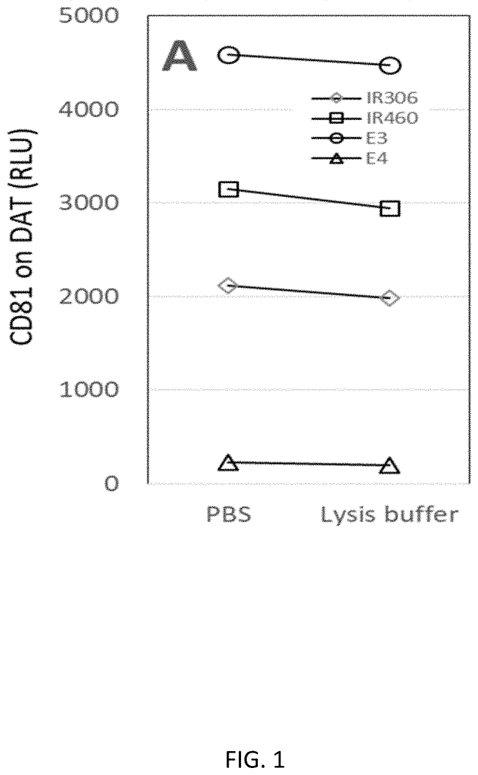

As shown in FIG. 1, CD81 signal was detected in wells treated with lysis buffer, demonstrating that exosomes were maintained on the ELISA plate even lysis buffer was applied.

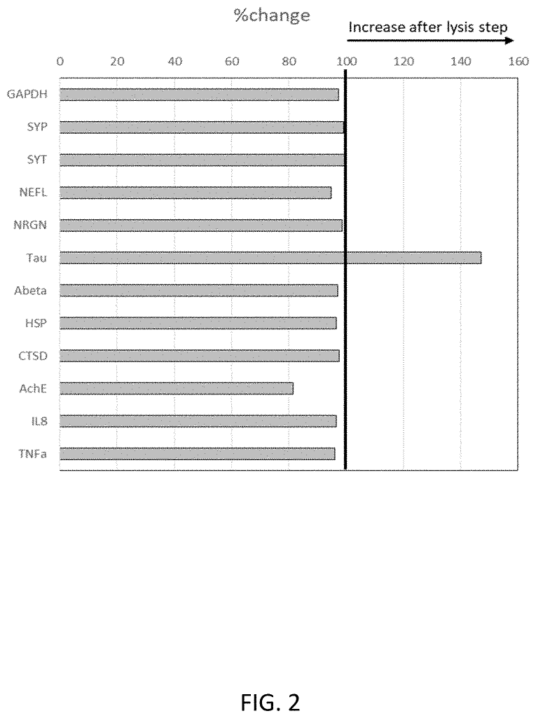

In another series of experiments, plasma samples were added to wells on an anti-SNAP25-immobilized ELISA plate to capture exosomes. After exosomes were captured, ELISA wells were exposed to PBS or lysis buffer prior to reacting with labeled antibodies against various biomarkers (See Y-axis in FIG. 2). Using the ELISA readings (RLU), percent change was calculated, with greater than 100% indicating that the biomarker was present in the inner membrane of the exosomes. As shown in FIG. 2, several exosomal biomarkers were unchanged, meaning that very low levels of these biomarkers can be found in the inner membranes of the exosomes. However, tau was significantly increased, indicating that tau is bound to exosome inner membranes.

These results showed that the methods of the present invention are useful for selectively capturing exosomes on a solid support, lysing or permeabilizing the exosomes while maintaining contact between the exosome membrane and the solid support, and detecting inner membrane-bound biomarkers from the captured exosomes.

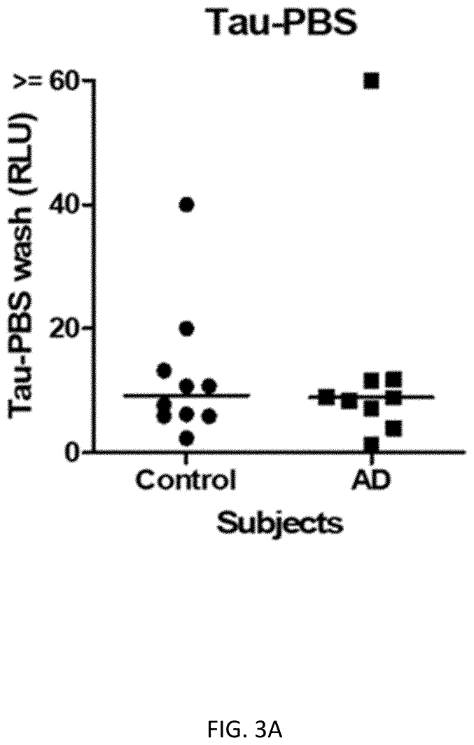

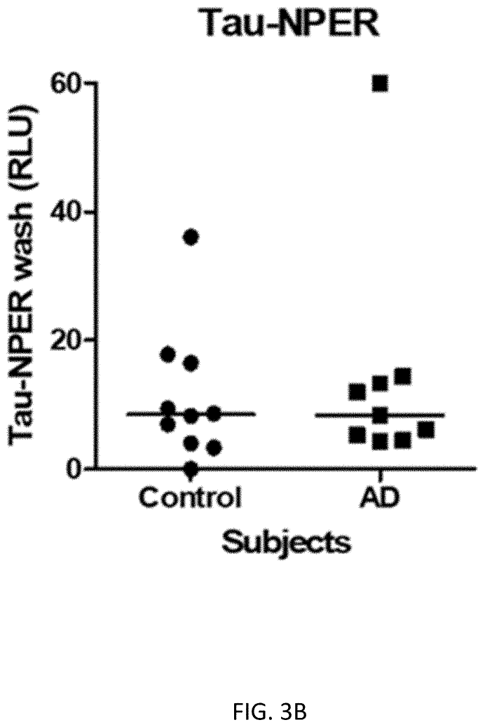

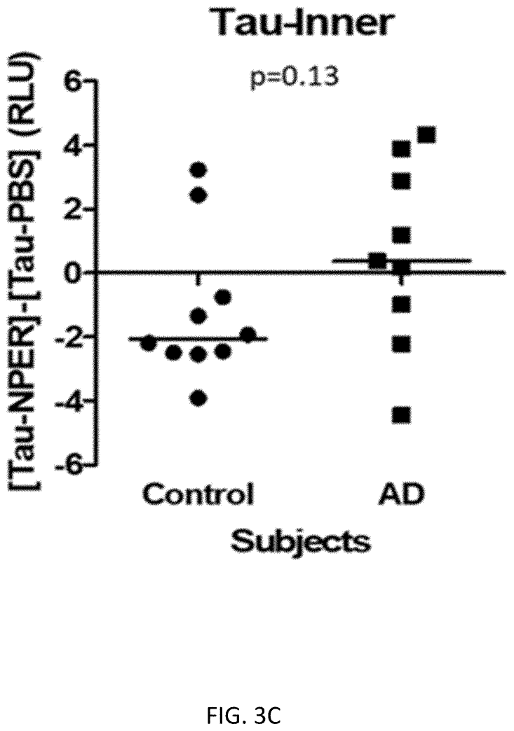

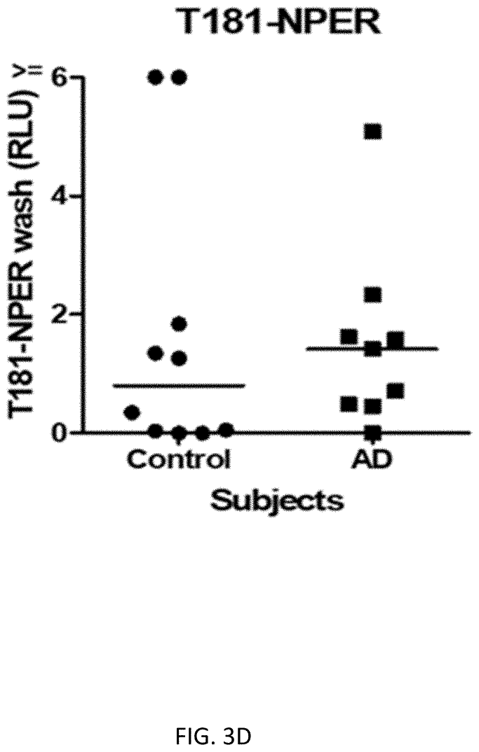

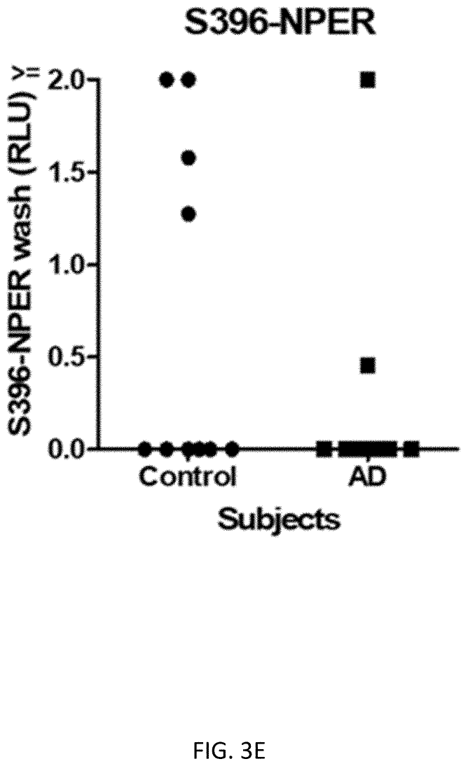

In another series of experiments, plasma samples were obtained from patients with Alzheimer's disease (AD). Plasma samples were added to wells on an anti-SNAP25-immobilized ELISA plate to capture exosomes. After exosomes were captured, ELISA wells were exposed to PBS or lysis buffer prior to reacting with biotinylated antibodies against Tau, phosphorylated tau T181, and phosphorylated tau S396. As shown in FIG. 3, several exosomal biomarkers were found in the inner membranes of the exosomes. Tau, T181 and S396 were all found in the inner membrane of the captured exosomes. Additionally, inner membrane-bound exosomal Tau levels were increased in samples from patients with Alzheimer's disease.

These results showed the detection of inner membrane-bound exosomal biomarkers. These results further showed that the methods of the present invention are useful for selectively capturing exosomes on a solid support, lysing or permeabilizing the exosomes while maintaining contact between the exosome membrane and the solid support, and detecting inner membrane-bound biomarkers from the captured exosomes. These results further showed that the methods of the present invention are useful for diagnosing neurological disease (e.g., Alheimer's disease).

### Example 2: Detection of Cytosolic Biomarkers from Exosomes

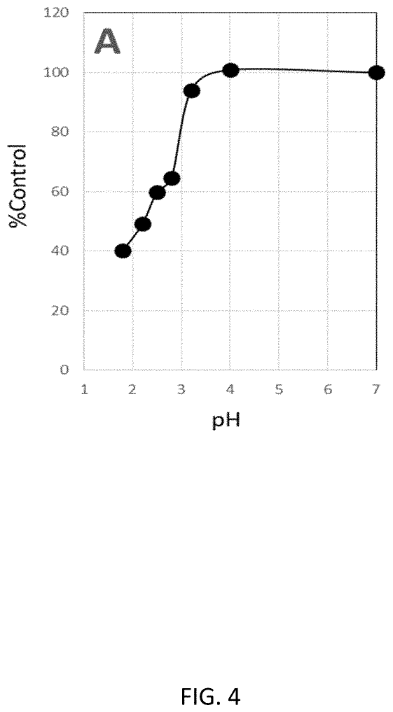

Cytosolic biomarkers were detected from exosomes as follows. Plasma samples were applied to anti-SNAP25-immobilized ELISA plates to capture exosomes. After exosomes were captured on the anti-SNAP25-immobilized ELISA plates, each well was washed with elution solutions with varying pH levels (from pH 7 to pH 1.8), followed by reaction with labeled anti-CD81 antibody. Results were expressed as % Control with the values at pH 7 as 100% and no plasma control as 0%. As shown in FIG. 4, ELISA signals were decreased by lowering pH, but even at pH1.8, approximately 40% of signals remained, suggesting the difficulty of eluting exosomes from the solid support.

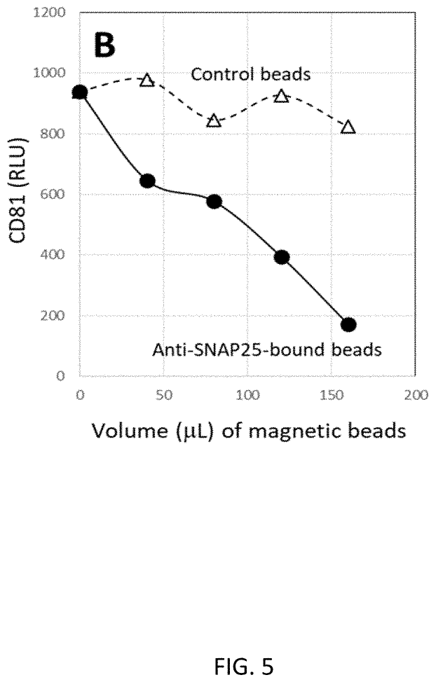

In another series of experiments, plasma samples were incubated with various volumes of magnetic beads with or without anti-SNAP25 antibody immobilization. Next, supernatants were applied to anti-SNAP25-immobilized ELISA plates followed by the reaction with labeled anti-CD81 antibody. As shown in FIG. 5, Anti-SNAP25-bound magnetic beads adsorbed SNAP25+CD81+ signals in a dose dependent manner, whereas control beads stayed unchanged, indicating capture of SNAP24+CD81+ exosomes by antibody-bound magnetic beads. Thus, by exposing lysis buffer to SNAP25-immobilized magnetic beads, whole exosome lysates can be obtained for analysis of cytosolic biomarkers.

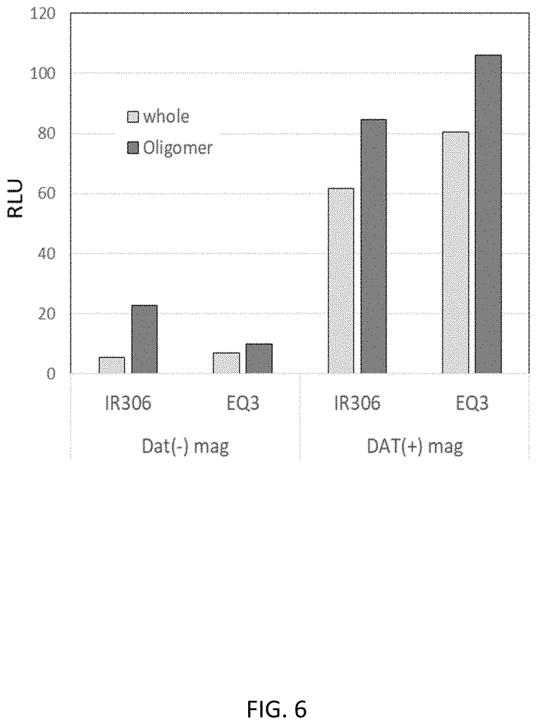

The □-synuclein (SNCA) is known as a pathological protein in Parkinson's disease (PD). In another series of experiments, exosomes were captured using the methods described above and cytosolic SNCA levels were determined as follows. Two plasma samples (IR306 and EQ3) were applied to magnetic beads with or without immobilization of anti-dopamine transporter (DAT) antibody (DAT(+) mag and DAT(−) mag, respectively). After washing extensively to remove any non-specifically bound materials, magnetic beads were exposed to lysis buffer. The resultant lysates were used for ELISA (monoclonal anti-SNCA immobilized ELISA plate, followed by another clone of labeled anti-SNCA antibody). As shown in FIG. 6, SNCA was detected in plasma samples applied to DAT(+) mag, but not from control beads DAT(−) mag.

These results showed that methods of the present invention are useful for detecting cytosolic biomarkers from exosomes. The results further demonstrated that the present invention is useful for selectively capturing exosomes on a solid support, lysing or permeabilizing the exosomes while maintaining contact between the exosome membrane and the solid support, and detecting cargo or cytosolic biomarkers from the captured exosomes.

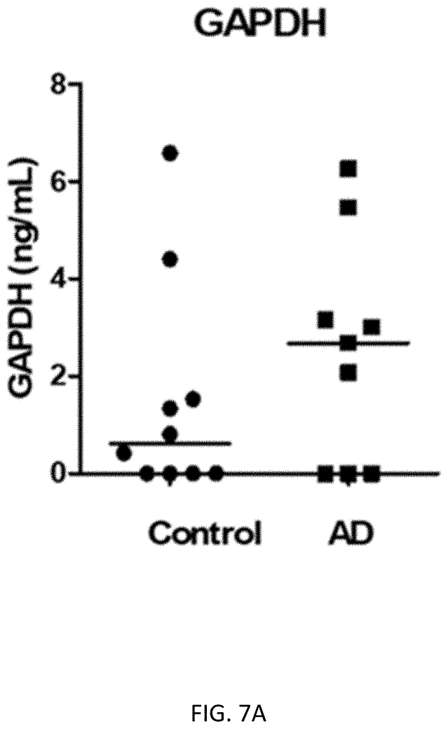

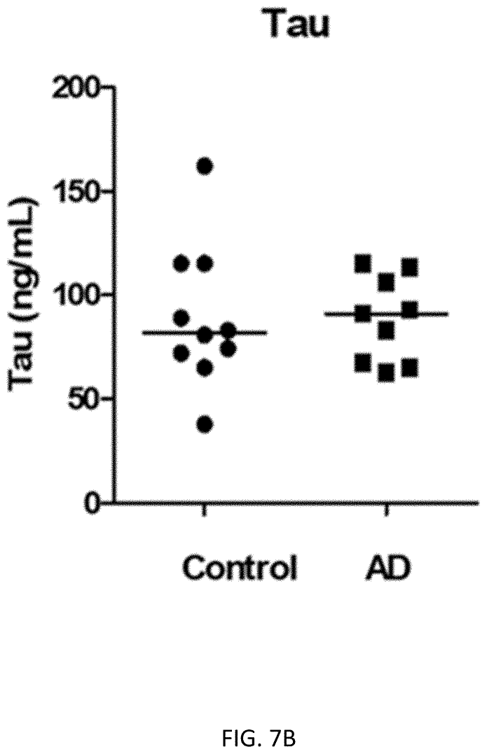

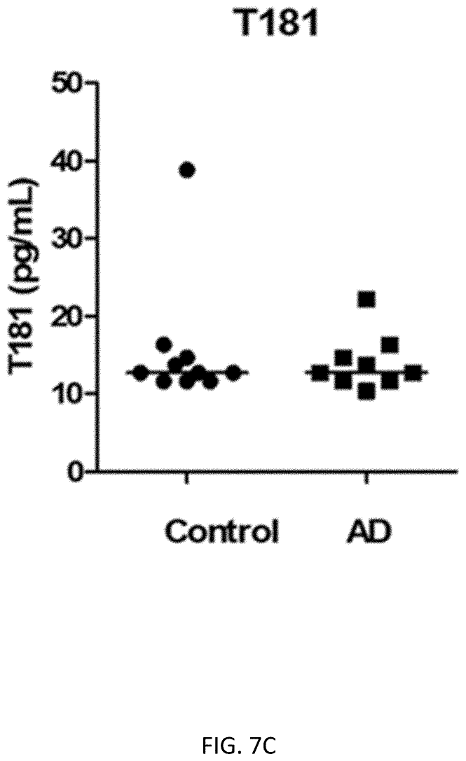

In another series of experiments, exosomes were captured and cytosolic proteins detected in plasma samples from patients with Alzheimer's disease (AD). AD and control plasma samples were applied to anti-SNAP25 immobilized ELISA plates to capture exosomes. After washing extensively to remove any non-specifically bound materials, ELISA plates were exposed to lysis buffer. The resultant lysates were used to quantify GAPDH, total tau, and p-tau T181 by ELISA, respectively. As shown in FIGS. 7A-7C, tau and T181 were detected in all samples, and due to low sensitivity, GAPDH was detected in 12 out of 19 samples. These data indicated that cytosolic exsomal cargo was successfully detected in samples from patients with Alzheimer's disease. These results showed that methods of the present invention are useful for detecting cytosolic biomarkers from exosomes. The results further demonstrated that the present invention is useful for selectively capturing exosomes on a solid support, lysing or permeabilizing the exosomes while maintaining contact between the exosome membrane and the solid support, and detecting cargo or cytosolic biomarkers from the captured exosomes.

### Example 3: Detection of miRNA from Exosomes

miRNA was detected from exosomes as follows. Plasma samples were incubated with anti-SNAP25-antibody magnetic beads and control antibody-free magnetic beads. After washing extensively to remove any non-specifically bound materials, magnetic beads were exposed to Trizol. The resultant lysates were used for RNA gel electrophoresis. miRNA was detected in plasma samples applied to anti-SNAP25-bound magnetic beads, but not from control antibody-free magnetic beads. These results showed that the methods of the present invention are useful for detecting miRNA from exosomes. These results further showed that methods of the present invention are useful for selectively capturing exosomes on a solid support, lysing or permeabilizing the exosomes while maintaining contact between the exosome membrane and the solid support, and detecting miRNA from the captured exosomes.

### Example 4: Saturation Enzyme-Linked Immunosorbent Assay to Detect Exosomal Biomarkers

Enzyme-linked immunosorbent assay (ELISA) is one of the most common immunoassays to detect and quantify the levels of various biomarkers. In order to quantify the levels of target biomarkers, the level of target analyte in the sample applied to the ELISA should be less than saturation amounts and stay within a linear range of the standard. There is no method or recommendation to intentionally apply saturable amounts of analytes in a sample to an ELISA.

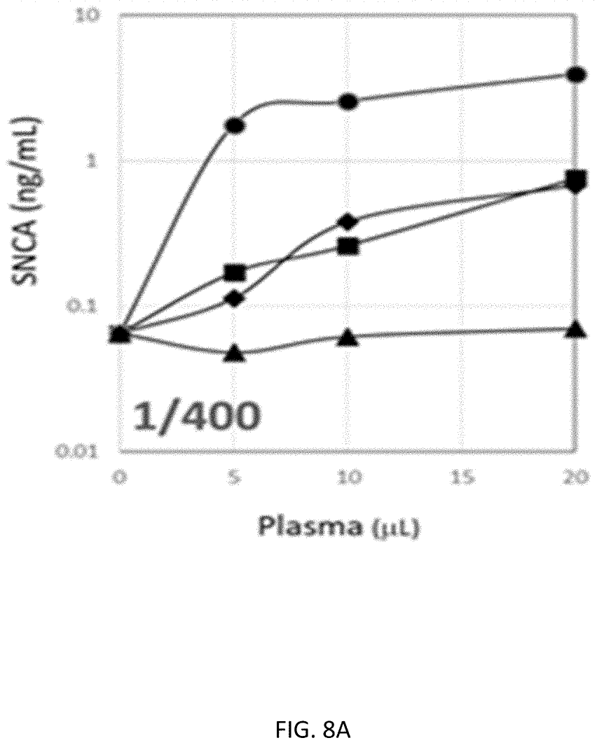

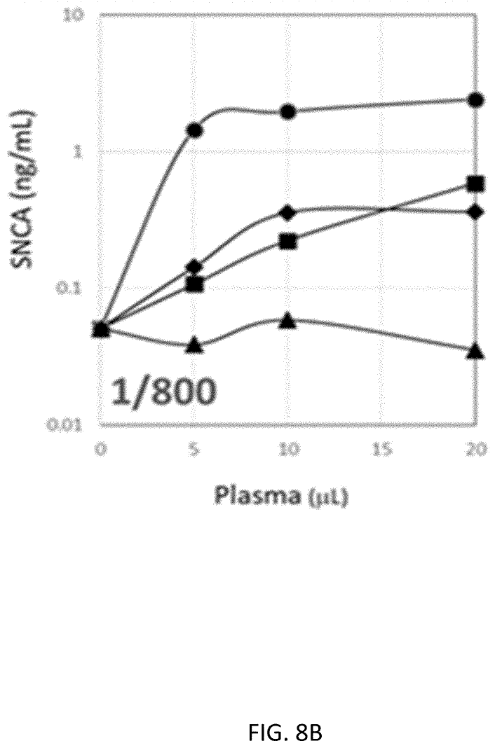

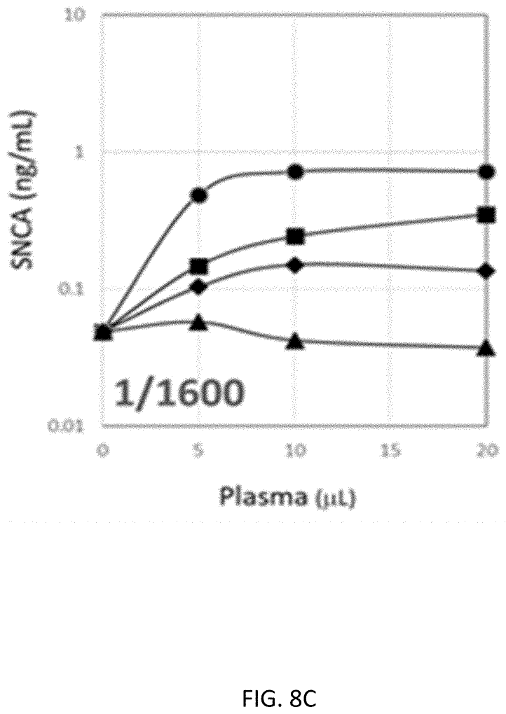

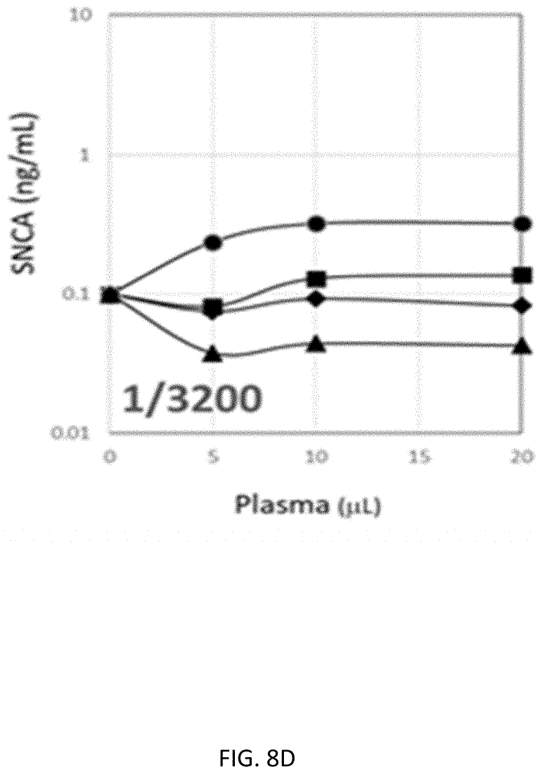

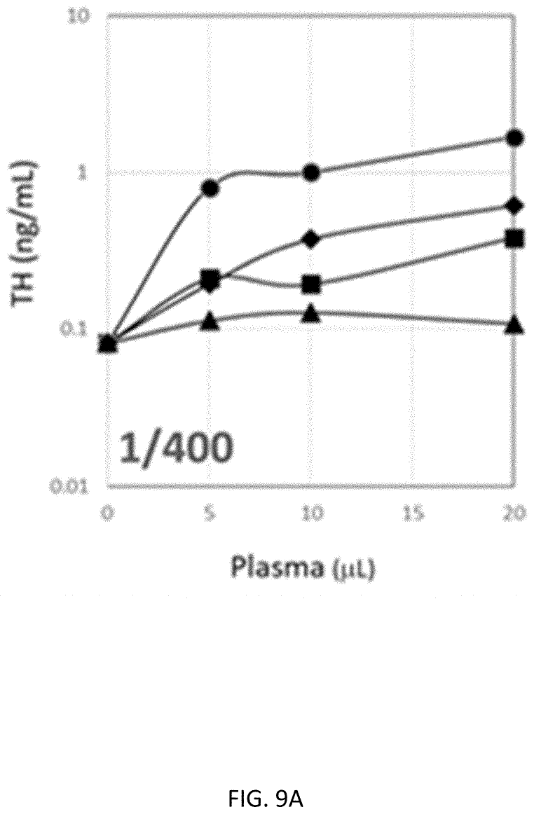

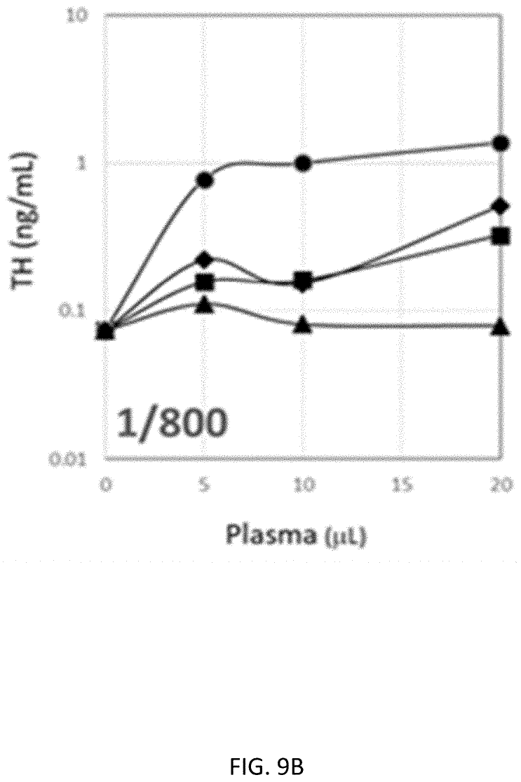

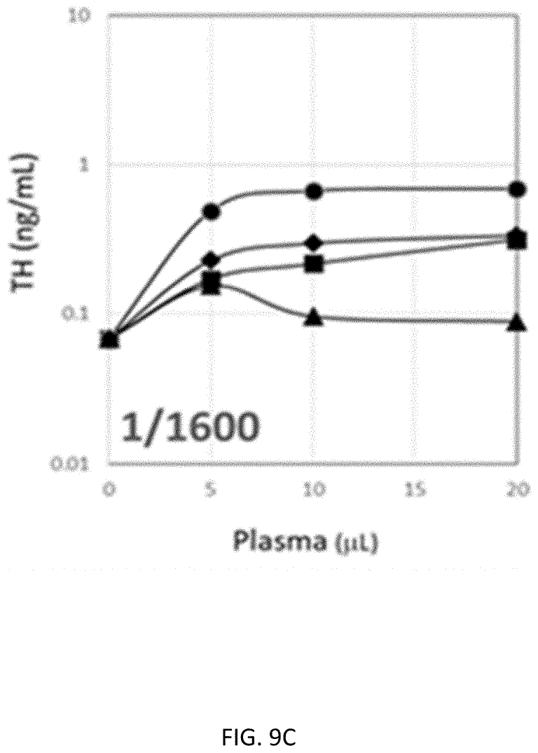

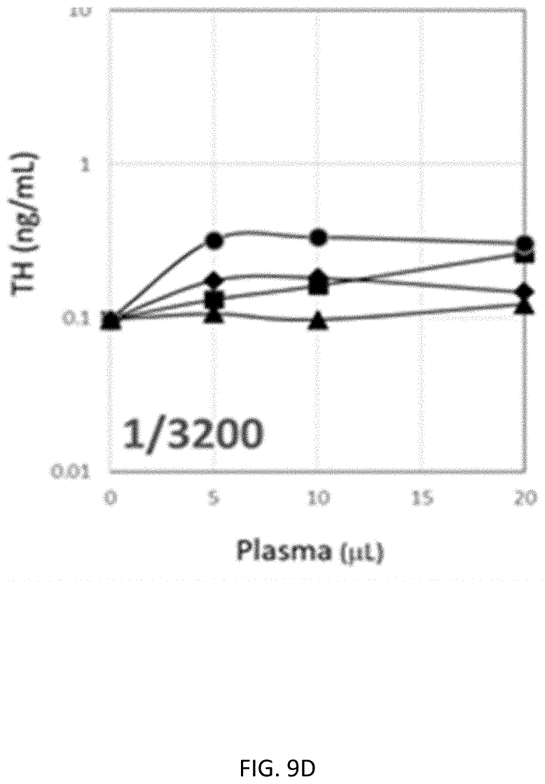

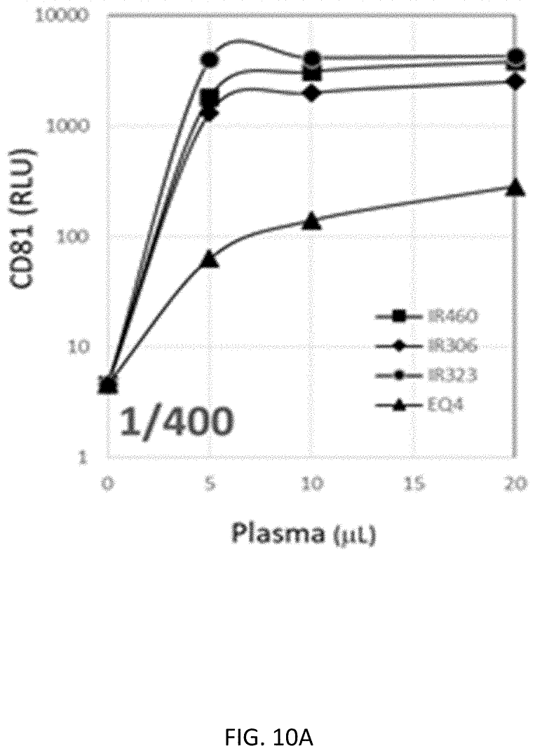

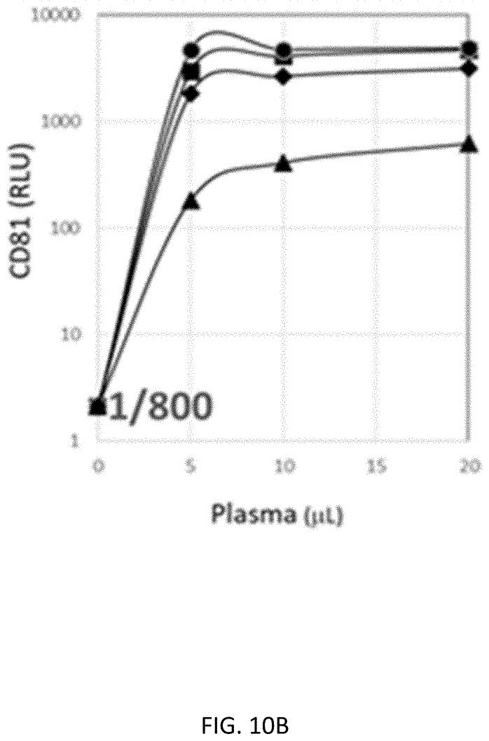

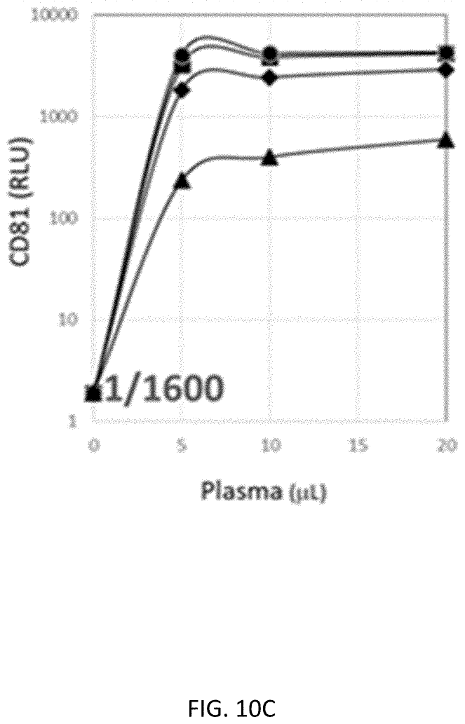

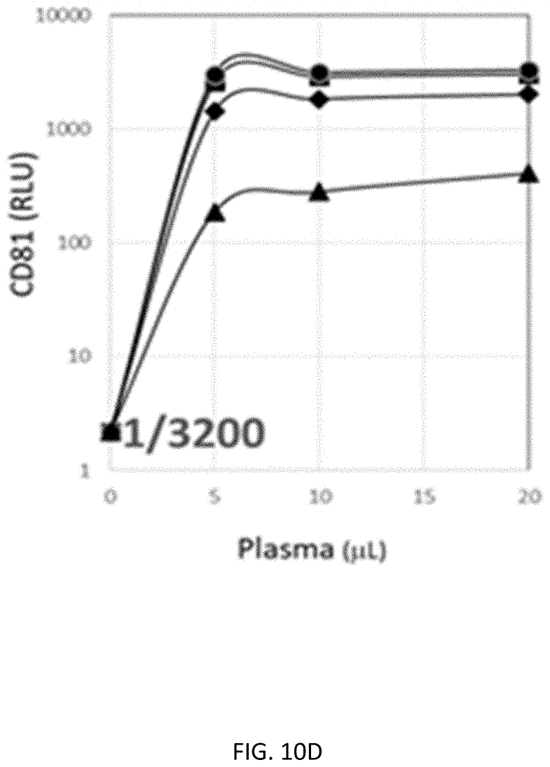

The quantity of immobilized antibodies for exosome saturation for the analysis of cargo proteins was determined as follows. Various volumes (20, 10, 5 and 0 uL) of four different plasma samples (IR460, IR306, IR323, and EQ4) were applied to ELISA plates, where antibody against dopamine transporter (DAT) was immobilized. The concentration of anti-DAT antibodies were 1/400 (=500 ng/mL), 1/800 (250 ng/mL), 1/1600 (125 ng/mL), and 1/3200 (62.5 ng/mL), respectively. After overnight incubation, each well was washed twice with wash buffer, then exposed to 50 uL lysis buffer supplemented with 1× each of both protease and phosphatase inhibitors for additional one hour at 4° C. Twenty microliters each of supernatants were transferred to ELISA kits of α-synuclein (SNCA) (FIGS. 8A-8D) and tyrosine hydroxygenase (TH) (FIGS. 9A-9D), respectively. Both SNCA and TH ELISA were conventional sandwich ELISA, not saturation ELISA. After lysates were transferred to SNCA and TH ELISA, each well was washed twice with wash buffer, then exposed to biotinylated anti-CD81 to quantify exosome membranes on anti-DAT plates (FIGS. 10A-10D).

As shown in FIGS. 10A-10D, the values of exosome membrane marker CD81 were saturated from 5-20 uL of 3 plasma (IR460, IR306, IR323) in all anti-DAT dilution (1/400-1/3200), whereas 10-20 uL EQ4 plasma was saturated from 1/800-1/3200 dilution, but not 1/400 dilution. The values of SNCA (FIGS. 8A-8D) and TH (FIGS. 9A-9D) in exosome cargo also showed that the values of 10 and 20 uL plasma were similar when anti-DAT antibodies were diluted more than 1/800, not 1/400, indicating saturation. However, the values of SNCA and TH were concentration dependent of anti-DAT antibody, and the values of 1/1600 and 1/3200 dilutions were lower than 1/800 dilution. Thus, optimal anti-DAT concentrations were 1/800 dilution (250 ng/mL).

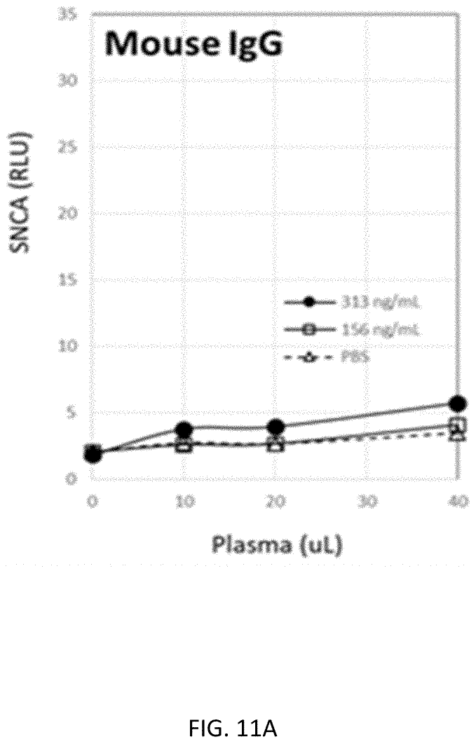

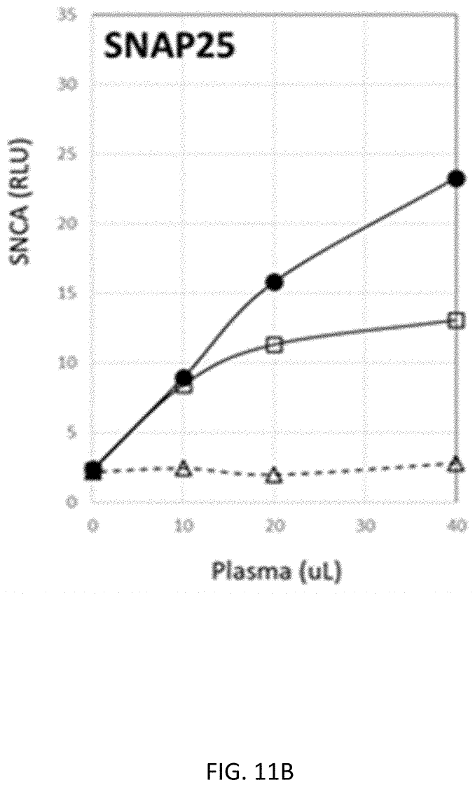

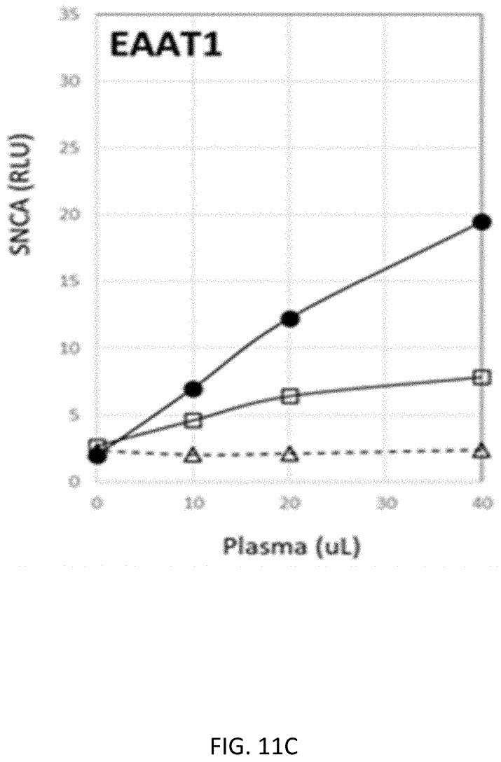

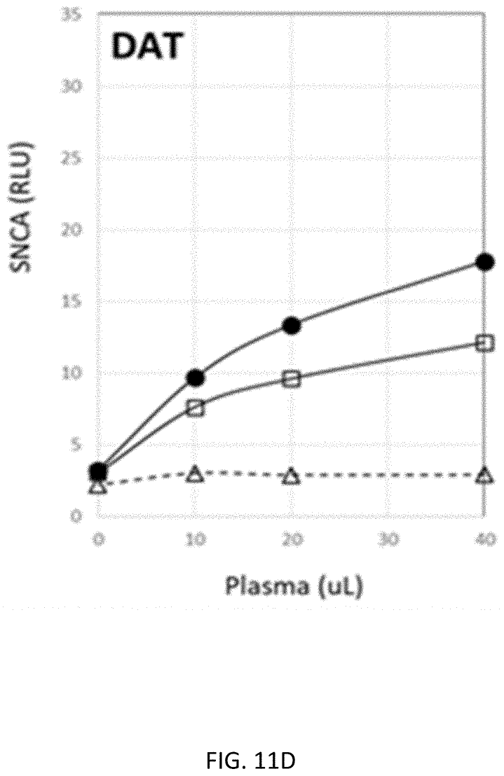

In another series of experiments, the quantity of various immobilized antibodies for exosome cargo protein analysis was determined. Various volumes (40, 20, 10, and 0 uL) of a plasma sample (IR460) were applied to ELISA plates, where 313 ng/mL, 156 ng/mL, and 0 ng/mL antibodies against mouse control IgG, synaptosomal-associated protein 25 (SNAP25, neuron marker), EAAT1, and DAT were immobilized. After exosomes were captured on ELISA plates, each well was exposed to lysis buffer to release cargo proteins. Next, the resultant lysates were transferred to separate SNCA ELISA plates. As shown in FIGS. 11A-11D, IR460 plasma was saturated between 20 and 40 mL on 156 ng/mL antibodies against SNAP25, EAAT1, and DAT, but not 313 ng/mL. Control mouse IgG were negative, indicating that the assay is antibody-specific.

These results showed that saturation enzyme-linked immunosorbent assays of the present invention are useful for detecting exosomes and exosomal biomarkers. These results further showed that the methods of the present invention are useful for incubating a sample comprising exosomes on a solid support comprising immobilized antibodies, wherein the immobilized antibodies on the solid support are generally saturated by the exosomes in the sample, and detecting biomarkers from the captured exosomes.

### Example 5: Isolation of Exosome Core

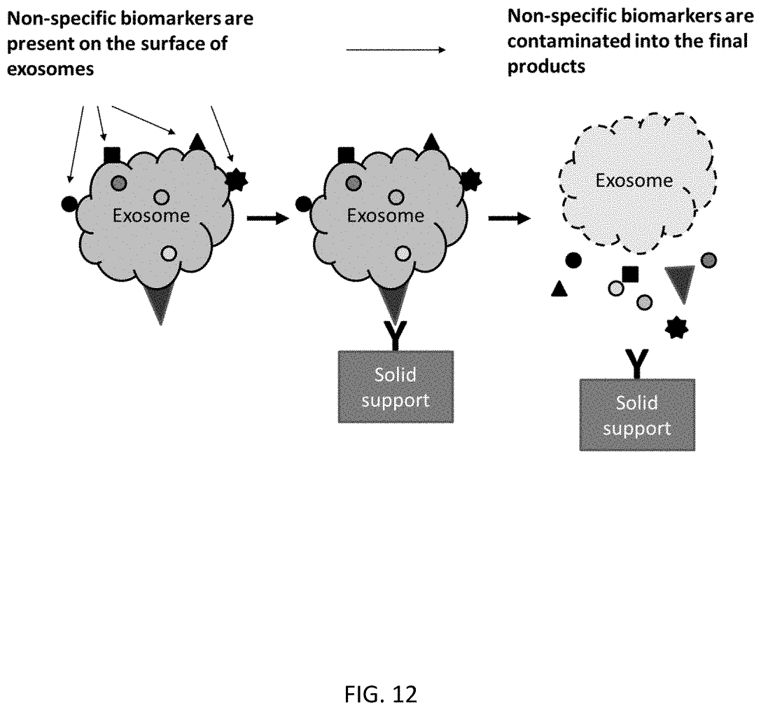

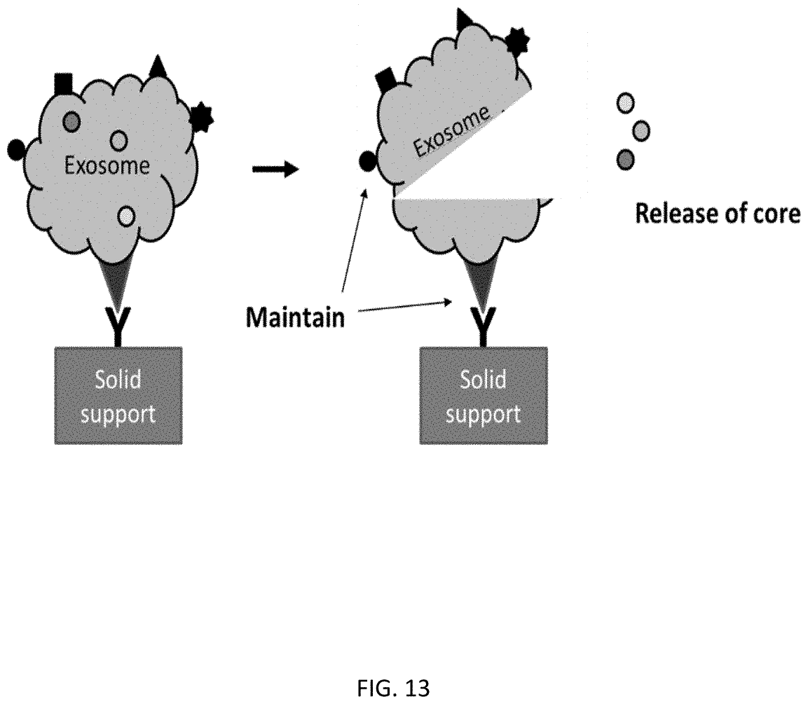

As shown in FIG. 12, standard ELISA assays used for exosome isolation can result in contamination with non-specific biomarkers in the final preparation. This limitation of standard ELISAs can be overcome by isolation of exosome core as described in FIG. 13. In this embodiment, exosomes were permeabilized while exosomes were bound on the solid support and exosome surface-bound biomolecules were also bound on the exosomes. The resultant core or cargo is mother cell-specific without (or minimum) contamination of foreign-origin biomarkers.

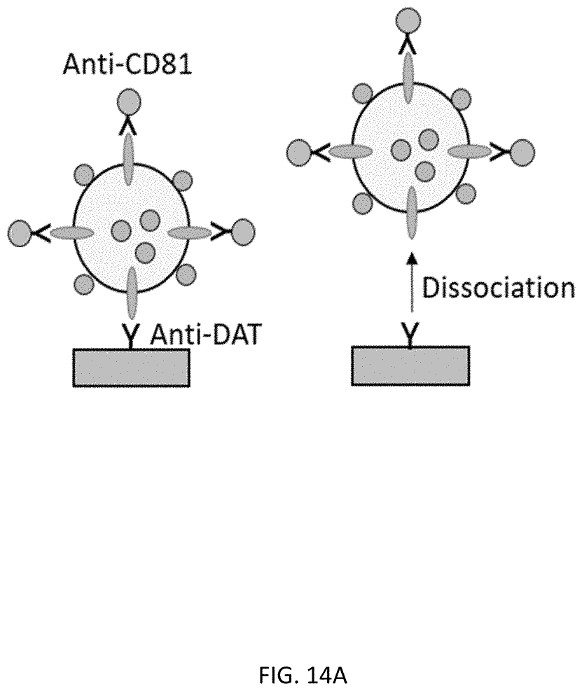

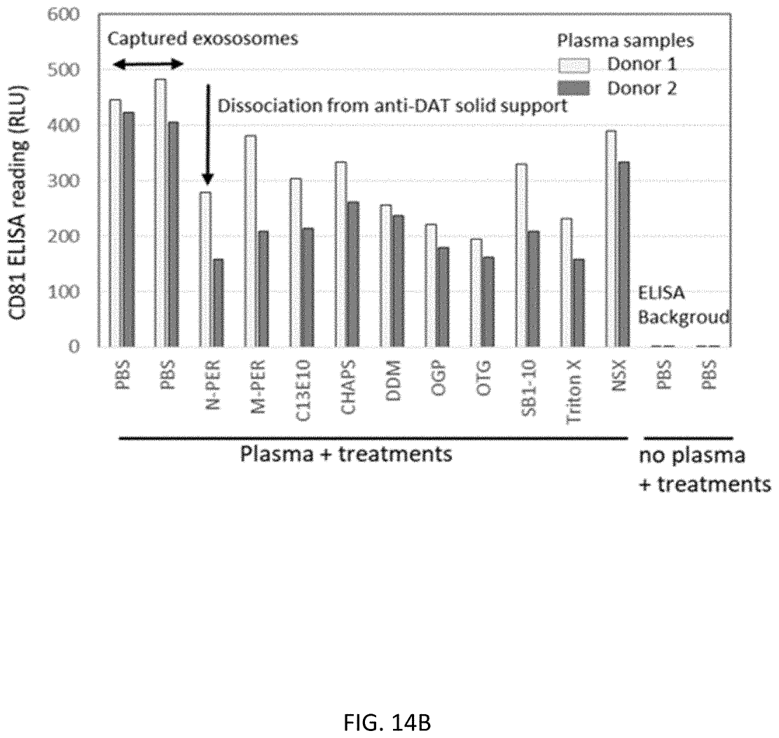

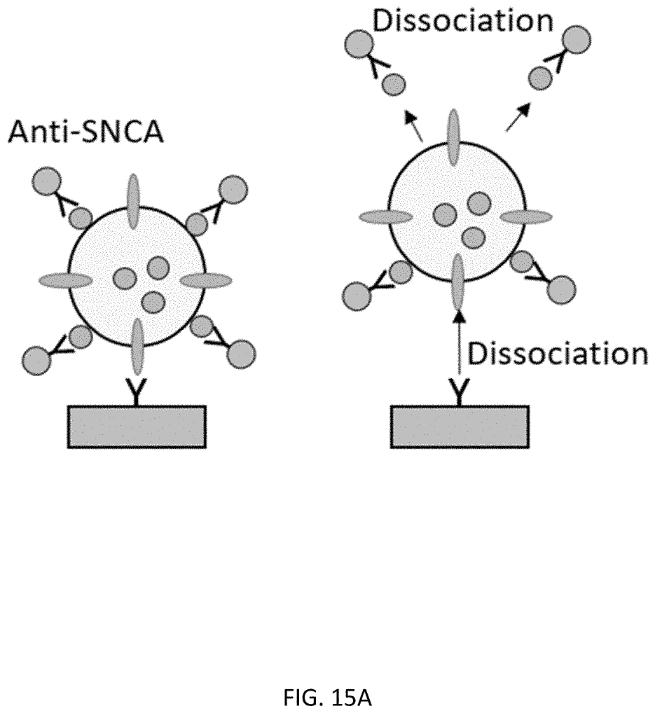

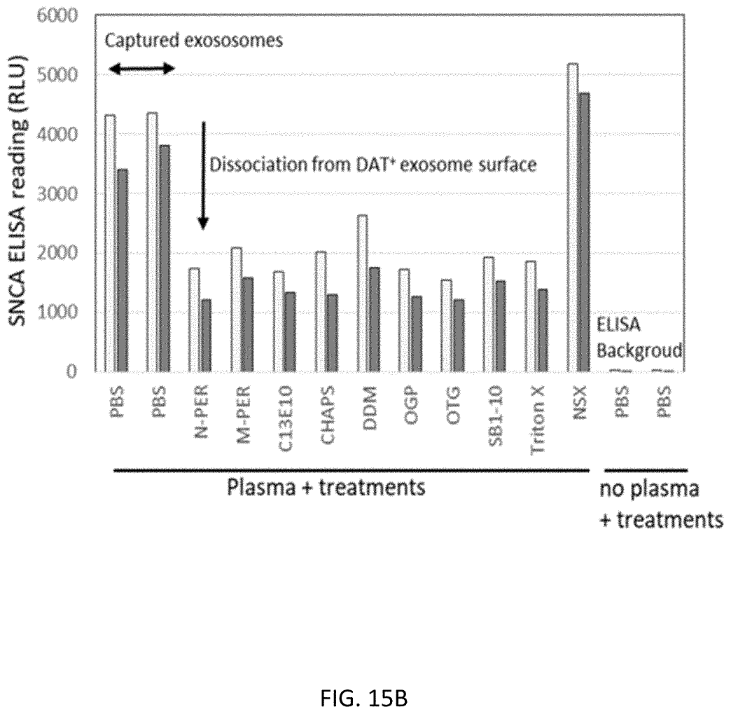

In one series of experiments, we demonstrate that exosomes stay on the solid support during core extraction. Dopaminergic neuron-derived exosomes were captured onto solid support, where anti-dopamine transporter (DAT) was immobilized. Multiple sets of two different donors' plasma samples were applied to a solid support to capture exosomes and unbound materials were washed away. One set of plasma samples was exposed to control buffer, phosphate buffered saline, and the other sets were exposed to various detergents, N-PER, M-PER, C13E10, CHAPS, DDM, OGP, OTG, SB1-1-, Triton-X, and NSX. After these detergents and PBS were washed, each solid support was reacted with biotinylated anti-CD81 (exosome common marker) followed by chemiluminescent ELISA to obtain relative light units (RLU) (see FIG. 15A). If exosomes are detached from the solid support, RLU should be decreased. As shown in FIG. 14B, all detergents decreased RLU indicating that exosomes were dissociated from the solid support. However, NSX maintained high RLU, indicating that exosome dissociation was minimal and that exosomes stay on the solid support during core extraction.

In another series of experiments, anti-CD81 was replaced with anti-□-synuclein (SNCA) to demonstrate the presence of SNCA on the surface of exosomes (see FIG. 15A). Similar to the example described above, if surface-bound SNCA is detached from the exosomes, RLU should be decreased.

As shown in FIG. 15B, all detergents decreased RLU indicating that SNCA was dissociated from the solid support. However, NSX maintained high RLU, indicating that SNCA was not dissociated from exosomes. Similar results were obtained by using anti-glyceraldehyde 3-phosphate dehydrogenase (GAPDH) (data not shown).

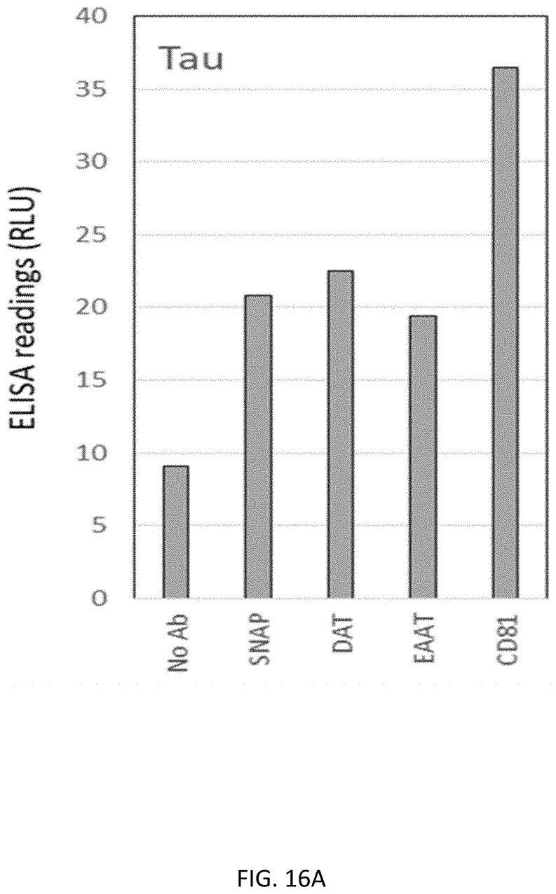

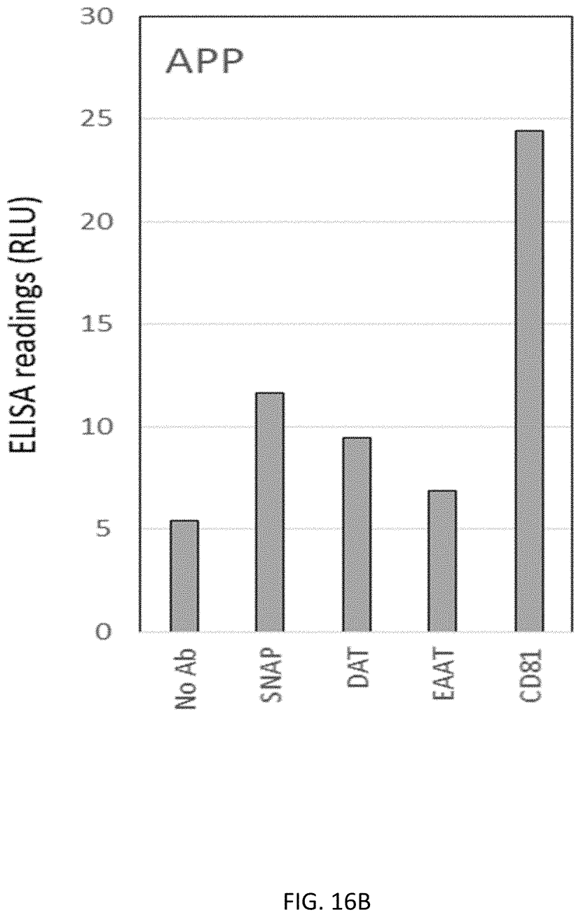

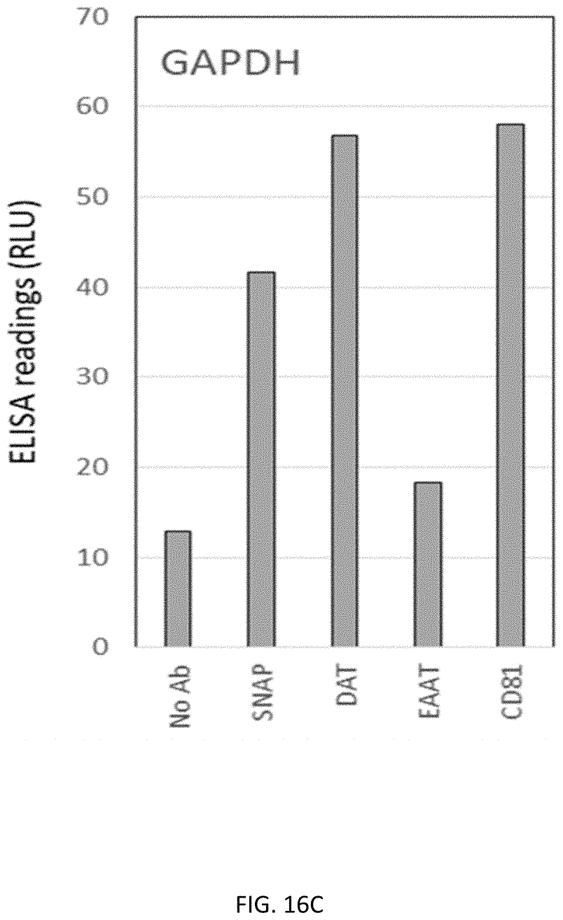

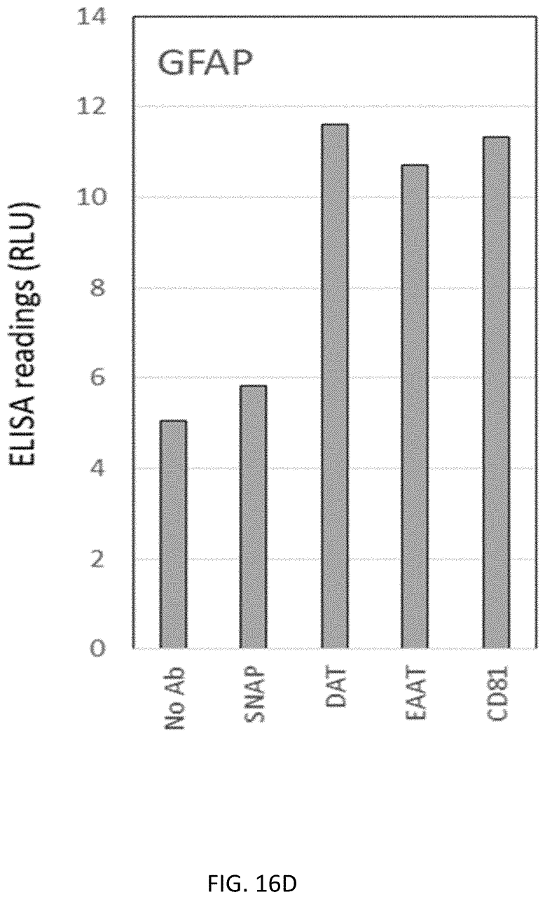

In the next FIG. 16A-D,

In another series of experiments, biomarkers from isolated exosome core were detected and measured as follows. Antibodies against SNAP25 (general neurons), DAT (dopaminergic neurons), EAAT1 (astrocyte markers), and CD81 (exosome common marker), as well as no antibody negative control were immobilized to magnetic beads, then exosome core samples were prepared by using NSX reagent. These core samples were immobilized onto ELISA wells, then probed with biotinylated antibodies against neuron marker Tau and amyloid precursor protein (APP), astrocyte marker GFAP, and positive control GAPDH. The GAPDH values (RLU) of SNAP25, DAT and CD81 were higher than those of no antibody control (see FIG. 16C), indicating that prepared core samples were not empty and contained at least positive control housekeeping GAPDH. The levels of GAPDH in astrocyte-derived exosomes (ADE) isolated by anti-EAAT1 was low, but slightly higher than negative control (see FIG. 16C). However, ADE showed high levels of astrocyte-specific GFAP protein, indicating that ADE core contained at least astrocyte-specific GFAP protein (see FIG. 16D). Similarly, neuron-derived exosomes (NDE) in both SNAP25 and DAT showed high levels of neuron-specific protein Tau and APP (see FIGS. 16A and 16B). These data clearly indicate that the exosome core prepared by our method contain mother-cell-derived biomarkers. These results demonstrated that the methods of the present invention are useful for maintaining contact between exosomes and solid supports, and between exosome membrane-bound biomarker and exosome membrane. These results further showed that the methods of the present invention are useful for isolating exosome core or cargo from the solid support. These results also showed that the methods of the present invention are useful for detecting cargo or cytosolic biomarkers from the exosome core.

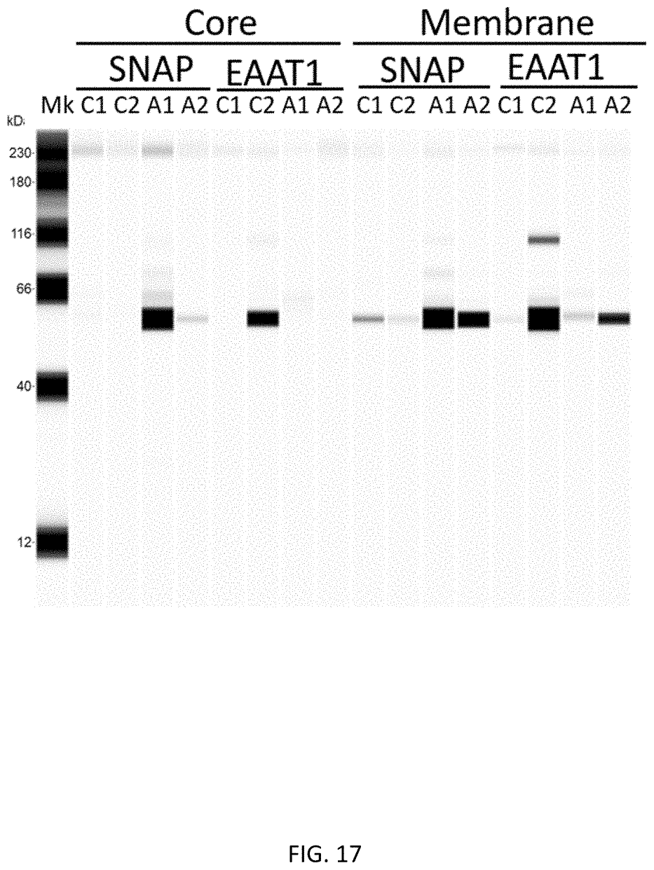

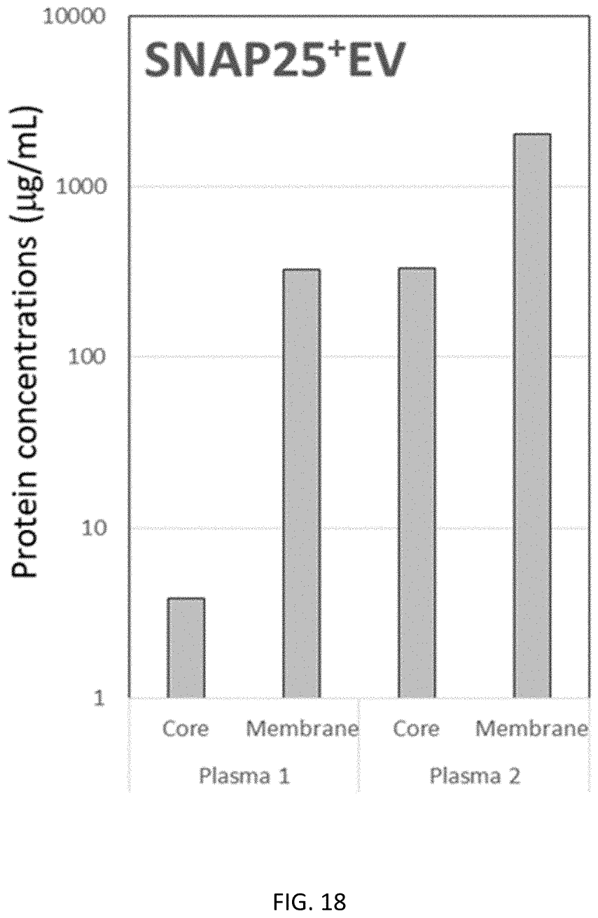

In another series of experiments, exosome core and membrane were prepared using two different capture antibodies against SNAP25 (NDE) and EAAT1 (ADE). Plasma samples were obtained from two human subjects with Alzheimer's disease (A1 and A2) and two healthy controls (C1 and C2). These samples were analyzed by Western blot probed with antibody against APP. The results are shown in FIG. 17. The molecular weight of intact APP is 110-135 KDa; we found only one sample with this molecular weight (C2 ADE membrane). The molecular weight of amyloid beta 1-42 is 4.5 KDa, and its oligomers are 40-200 KDa. As shown in FIG. 17, a 58-63 KDa band was present in the Western blots from three core samples and from all eight membrane samples. These results indicate that core and membrane in both NDE and ADE contain amyloid oligomers, and that the quantity is greater in membrane compared to core.

In another series of experiments, dopamine levels in exosome core samples was determined as follows. Exosome core samples were prepared using anti-SNAP25 and anti-DAT-immobilized magnetic beads. Dopamine levels were determined by mass spectrometry. As shown in Table 1 below, exosome core samples prepared from anti-SNAP25 and anti-DAT-immobilized magnetic beads contained dopamine. Conversely, no dopamine was detected in samples prepared by control antibody magnetic beads. These results indicated that neuron-derived exosome (NDE) core contains the neurotransmitter dopamine. These results further showed that the SNAP25+ exosomes were derived from neurons.

TABLE 1
Quantification of neurotransmitter dopamine in exosome core.

| | | Magnetic beads | | |
|---|---|---|---|---|
| Neurotransmitter | Buffer | No Ab | SNAP | DAT |
| Dopamine | 0 | 0 | 1593 | 654 |

In another series of experiments, total protein concentration in both SNAP25+ exosome membranes and core was determined as follows. SNAP25+ exosome membranes and core were isolated by magnetic beads as described above. Total protein concentrations were determined by micro BCA method in both SNAP25+ exosome membranes and core samples. An exosome membrane fraction was prepared by adding 0.5% SDS after exosome core was released. Both NSX and SDS did not influence micro BCA assay. The quantity was very different between two donor samples, but protein content in the membrane fraction was 10 to 100-fold greater than core. These results indicated that the biomarkers of the exosome membrane biomarkers contaminate and/or mask the biomarker content in the exosome core.

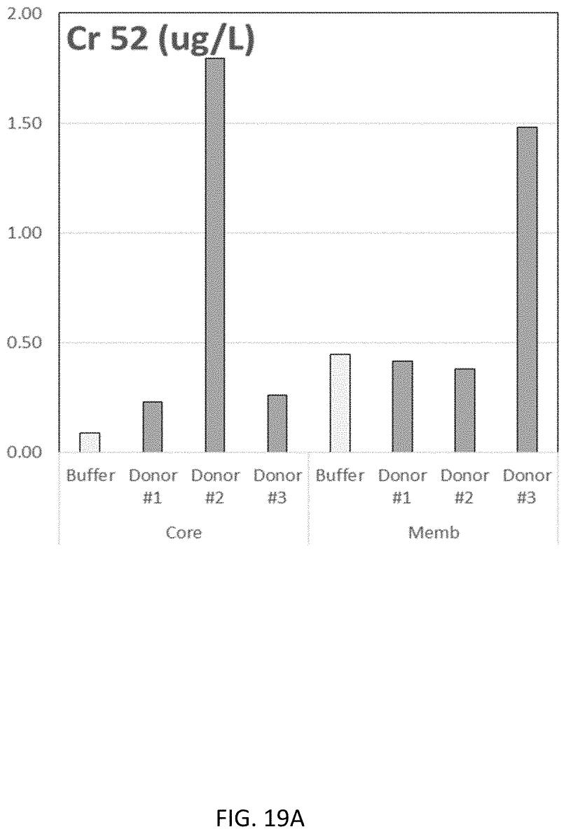

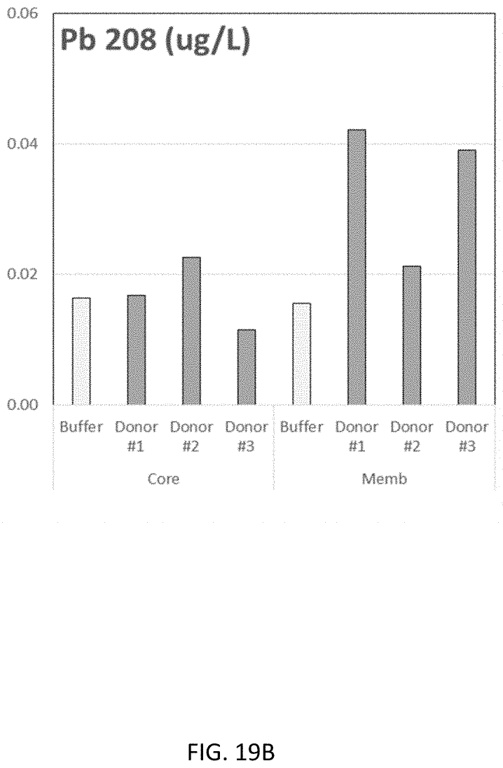

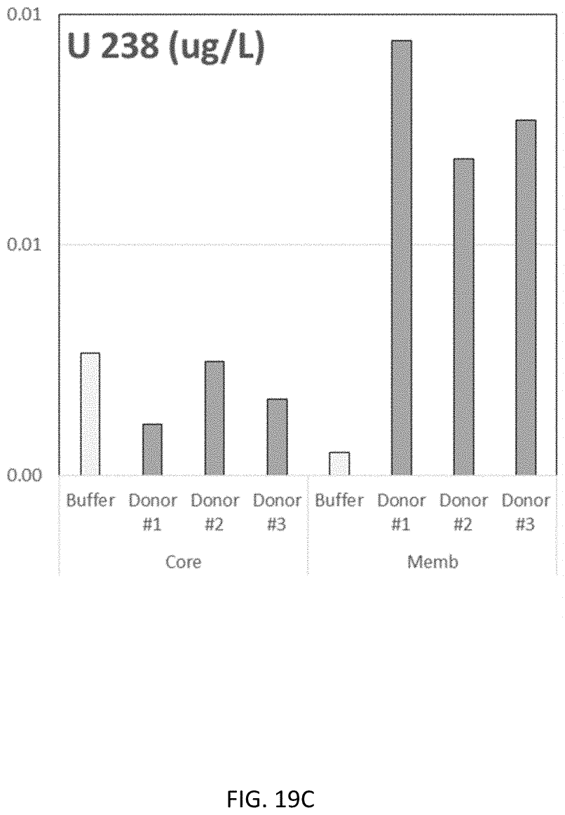

In another series of experiments, heavy metal content in exosome membrane and exosome core was determined. Plasma samples were obtained from three different donors. SNAP25+ exosome core and membrane fractions were prepared as described above. The prepared fraction samples were applied to mass spectrometry to analyze heavy metal. Although samples processed with NSX buffer and SDS contained some metals (light gray bars in FIGS. 19A-19C), Cr in exosome core were higher than NSX buffer alone, whereas Pb and U238 in the exosome core were not higher than NSX buffer (see FIGS. 19A-19C). In contrast, Pb and U238 levels were higher than SDS buffer alone in all three donor samples, indicating that NAP25+ NDE membranes contain Pb and U238. However, Cr was only positive in one of the three membrane fraction samples. These results showed that exosome core and membrane fractions were applicable to mass spectrometry for metal analysis. The results also showed that heavy metal levels in exosome core are not identical to the levels in the exosome membrane and that membrane composition is different than core composition. These results also showed that the methods of the present invention are useful for detecting cargo or cytosolic biomarkers from the exosome core.

Various modifications of the invention, in addition to those shown and described herein, will become apparent to those skilled in the art from the foregoing description. Such modifications are intended to fall within the scope of the appended claims. These results showed that the methods and compositions of the present invention are useful for identifying, detecting, and measuring biomarkers from exosomes captured on a solid support, wherein the captured exosomes are lysed or permeabilized and contact between the exosome membrane and the solid support is maintained. These results further suggested that the methods and compositions of the present invention would be useful for diagnosing a disease or disorder in a subject.

All references cited herein are hereby incorporated by reference herein in their entirety.

What is claimed is:

1. A method comprising: a) providing a biological sample comprising vesicles; b) contacting a solid support comprising capture agents associated therewith with the biological sample under conditions wherein the capture agents selectively bind to the vesicles, thereby capturing said vesicles on the solid support; c) lysing or permeabilizing the vesicles while maintaining contact between the vesicle and the solid support, and between vesicle membrane-bound biomarker and vesicle membrane; and d) isolating vesicle core from the captured vesicles.

2. The method of claim 1, wherein the biological sample is selected from the group consisting of whole blood, serum, plasma, urine, interstitial fluid, peritoneal fluid, tears, saliva, cerebrospinal fluid, cell and/or bacterial culture supernatants, cervical swab, buccal swab, tissues, organs, and environmental materials.

3. The method of claim 1, wherein the vesicles are selected from the group consisting of exosomes, microparticles, microvesicles, nanosomes, extracellular vesicles, ectosomes, and apoptotic bodies.

4. The method of claim 3, wherein the exosomes are selected from the group consisting of neuron-derived exosomes, astrocyte-derived exosomes, oligodendrocyte-derived exosomes, microglia-derived exosomes, dopaminergic neuron-derived exosomes, cholinergic neuron-derived exosomes, serotonergic neuron-derived exosomes, histaminergic neuron-derived exosomes, glutaminergic neuron-derived exosomes, glycinergic neuron-derived exosomes, adrenergic neuron-derived exosomes, gabaergic neuron-derived exosomes, opipoidergic neuron-derived exosomes, cancer-derived exosomes, bone marrow-derived exosomes, lymph node-derived exosomes, prostate-derived exosomes, lung-derived exosomes, liver-derived exosomes, pancreas-derived exosomes, ovary-derived exosomes, gastrointestinal-derived exosomes, kidney and urinary tract-derived exosomes, skin-derived exosomes, bone-derived exosomes, muscle-derived exosomes, peripheral nerve-derived exosomes, adipose tissue-derived exosomes, connective tissue-derived exosomes, male or female organ-derived exosomes, spleen-derived exosomes, endocrine-derived exosomes, and vascular-derived exosomes.

5. The method of claim 1, wherein the solid support is a plate, a non-magnetic bead, a magnetic bead, a filter, a slide, a wafer, a rod, a particle, a strand, a disc, a membrane, or a surface of a tube, channel, column, flow cell device, pipette tip, or microfluidic device.

6. The method of claim 1, wherein the solid support comprises glass, quartz, silicon, metal, ceramic, plastic, nylon, polyacrylamide, agarose, a hydrogel, or a resin.

7. The method of claim 1, wherein the capture agents are antibodies, antibody fragments, antibody mimetics, aptamers, receptors, or ligands that specifically bind to the vesicles.

8. The method of claim 7, wherein the antibodies are monoclonal or polyclonal antibodies against CD81, CD63, CD9, CD171, NCAM, SNAP25, EAAT1, OMG, CD11b, Tau, amyloid beta, alpha-synuclein, TDP-43, and/or SOD.

9. The method of claim 1, further comprising detecting one or more cargo and/or cytosolic biomarkers from the vesicle core.

10. The method of claim 9, wherein the biomarker is a mutated protein, an over- or under expressed protein, a modified protein, an organ/tissue-specific protein, a disease-associated protein, a protein monomer and/or oligomer, an enzyme, a kinase, a hormone, a growth factor, a transcription factor, a cytokine and/or chemokine, miRNA, mRNA, non-coding RNA, a neurotransmitter, a small molecule, a metabolite, a lipid and/or lipoprotein, a metal, a foreign body, and/or control markers.

11. The method of claim 10, wherein the modified protein is phosphorylated protein, methylated protein, glycosylated protein, acetylated protein, or ubiquitinated protein.

12. The method of claim 10, wherein the disease-associated proteins are selected from the group consisting of a monomer, oligomer, or phosphorylated form of tau, amyloid beta, alpha-synuclein, TDP-43, SOD.

13. The method of claim 10, wherein the control markers comprise total protein, synaptic proteins, GAPDH, GFAP, and MBP.

14. The method of claim 9, wherein the detection comprises immunoassay, Western blot, Northern blot, chromatography, mass-spectrometry, sequencing, and/or reaction with nucleic acid dye.

15. The method of claim 1, wherein the biological sample is obtained from a subject who has been diagnosed or is suspected of having a neurological disorder, Alzheimer's disease (AD), vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), stroke, depression, bipolar disease, epilepsy, autism, schizophrenia, brain tumor, white matter disease, brain atrophy, mental retardation, cerebellar ataxia, multiple system atrophy, Parkinson's disease.

16. The method of claim 1, wherein the biological sample is obtained from a subject who has been diagnosed or is suspected of having breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, or spinal axis tumors.

17. The method of claim 9, further comprising treating the subject for a disease or disorder if the subject is diagnosed as having the disease or disorder.

18. A method comprising: a) providing a biological sample comprising exosomes; b) contacting a solid support comprising capture agents associated therewith with the biological sample under conditions wherein the capture agents selectively bind to the exosomes, thereby capturing said exosomes on the solid support; c) lysing or permeabilizing the exosomes while maintaining contact between the exosome membrane and the capture agents; and d) detecting inner membrane-bound biomarkers from the exosomes.

19. The method of claim 18, wherein the inner membrane-bound biomarker is selected from the group consisting of a monomer, oligomer, phosphorylated form of tau, synaptophysin, synaptotagmin, synaptopodin, SNAP25, neurofilament, amyloid beta, alpha-synuclein, TDP-43, SOD aptic protein, a cytoskeletal protein, a membrane receptor associated protein and/or kinase.

20. The method of claim 18, wherein the exosomes are selected from the group consisting of neuron-derived exosomes, astrocyte-derived exosomes, oligodendrocyte-derived exosomes, microglia-derived exosomes, dopaminergic, cholinergic, serotonergic, histaminergic, glutaminergic, glycinergic, adrenergic, gabaergic, and opipoidergic neuron-derived exosomes, cancer-derived exosomes, bone marrow-derived exosomes, lymph node-derived exosomes, prostate-derived exosomes, lung-derived exosomes, liver-derived exosomes, pancreas-derived exosomes, ovary-derived exosomes, gastrointestinal-derived exosomes, kidney and urinary tract-derived exosomes, skin-derived exosomes, bone-derived exosomes, muscle-derived exosomes, peripheral nerve-derived exosomes, adipose tissue-derived exosomes, connective tissue-derived exosomes, male or female organ-derived exosomes, spleen-derived exosomes, endocrine-derived exosomes, and vascular-derived exosomes.
